(19) United States
(12) Patent Application Publication
(10) Pub. No.: US 2016/0318892 A1
(43) Pub. Date: Nov. 3, 2016

(54) PRODRUG COMPOUNDS

(71) Applicant: PROXIMAGEN LIMITED, Cambridge (GB)

(72) Inventors: Edward Savory, Cambourne (GB); Martyn Pritchard, St. Ives (GB); Daniel C. Hill, Maple Grove, MN (US)

(21) Appl. No.: 15/107,616

(22) PCT Filed: Dec. 22, 2014
(86) PCT No.: PCT/GB2014/053816

§ 371 Date: Jun. 23, 2016

(30) Foreign Application Priority Data
Dec. 23, 2013 (GB) 1322934.9

(51) Int. Cl. C07D 311/68; C07D 409/12; C07D 405/12

(52) U.S. Cl. CPC C07D 311/68; C07D 405/12; C07D 409/12

(57) ABSTRACT

A compound of formula (Ia) or (Ib), or a pharmaceutically acceptable salt thereof, Wherein Q, R², Ar, A and R¹ are as defined in claim 1. The claimed compounds are gap junction blockers useful for the treatment or prevention of a range of conditions including migraine, epilepsy, non-epileptic seizures, brain injury (including stroke, intracranial haemorrhage and trauma induced), pain, neurodegenerative disease or cardiovascular disease including myocardial infarction, coronary revascularization or angina.

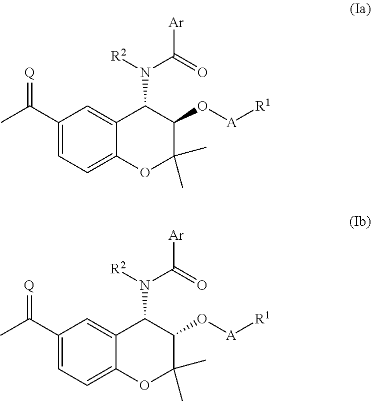

The present invention relates to pharmaceutically active compounds having improved pharmacokinetic properties, the compounds being useful for the treatment or prevention of a range of conditions including migraine, epilepsy, non-epileptic seizures, brain injury (including stroke, intracranial haemorrhage and trauma induced) or cardiovascular disease including myocardial infarction, coronary revascularization or angina.

### BACKGROUND TO THE INVENTION

Cortical spreading depolarization (CSD) is a wave of depolarisation with consequent depressed electrical activity which spreads across the surface of the cerebral cortex (at a rate of 2-6 mm/min) usually followed by hyperaemia and neuronal hyperpolarisation. The reduction in electrical activity is a consequence of neuron depolarisation and swelling, with K+ efflux, Na and Ca influx and electrical silence. This abnormal neuronal activity is associated with delayed neuronal damage in a number of pathological states including cerebral ischaemia (arising from e.g. stroke, haemorrhage and traumatic brain injury Strong et al., 2002 Fabricius et al., 2006; Dreier et al., 2006 Dohmen et al., 2008), epilepsy and the aura associated with migraine (Lauritzen 1994; Goadsby 2007). As the CSD wave moves across the cortex it is associated with a reactive increase in local blood flow which may serve to help restore the more normal ionic balance of the neurons affected. After the CSD induced hyperaemia the local increase in blood flow attenuates (oligaemia) potentially resulting in imbalances in energy supply and demand. Under certain conditions, the reactive hyperaemia is not observed, but instead the local vasculature constricts resulting in ischaemia which in turn can lead to neuronal death. The conditions triggering this abnormal response in experimental models are high extracellular levels of K+ and low NO availability. These conditions are typically seen in ischaemic areas of the brain, and clusters of CSD waves in these circumstances result in spreading ischaemia (see Dreier 2011). Of particular importance is the spreading ischaemia seen after sub-arachnoid haemorrhage (SAH), in the penumbra of an infarct and after traumatic brain injury where delayed neuronal damage can have a significant effect on clinical outcomes (Dreier et al., 2006, 2012; Hartings et al., 2011a, 2011b; Fabricius et al., 2006).

Given the detrimental effect of clusters of CSDs in humans and experimental animals, and the poor prognosis associated with CSDs, there is an unmet medical need for new compounds useful for inhibiting CSDs for patients with and without brain injuries. Without wishing to be bound by theory, the spread of CSD is believed to be mediated by gap junctions rather than by neuronal synaptic communication (Nedergard et al., 1995; Rawanduzy et al., 1997, Saito et al., 1997), the gap junctions providing a means of spreading the depolarisation in the absence of normal synaptic communication. Gap junctions are comprised of connexin proteins of which there are 21 in the human genome. Each Gap junction is made of two hemichannels, each comprising six connexin monomers.

Gap junctions are also implicated in a number of other disease states including hereditary diseases of the skin and ear (e.g. keratitis-ichthyosis deafness syndrome, erythrokeratoderma variabilis, Vohwinkel's syndrome, and hypotrichosis-deafness syndrome). Blockade of gap junction proteins has been shown to beneficial in some preclinical models of pain (e.g. Spataro et al., 2004 J Pain 5, 392-405, Wu et al., 2012 J Neurosci Res. 90,337-45). This is believed to be a consequence of gap junction blockade in the spinal cord resulting in a reduction in the hypersensitivity of the dorsal horn to sensory nerve input. In addition gap junctions and their associated hemichannels have been implicated in neurodegenerative diseases including Alzheimer's disease, Parkinson's Disease, Huntington's Disease and amyotrophic lateral sclerosis (Takeuchi et al 2011 PLoS One.; 6, e21108).

Tonabersat (SB-220453/PRX201145) is a gap junction blocker (Silberstein, 2009; Durham and Garrett, 2009) which binds selectively and with high affinity to a unique stereo-selective site in rat and human brains. Consistent with its action on gap junctions Tonabersat also inhibits high K+ evoked CSD in cats (Smith et al., 2000; Read et al., 2000; Bradley et al., 2001) and rats (Read et al., 2001).

However, known gap junction blockers, including Tonabersat and Carabersat, suffer from undesirable physiochemical properties. Tonabersat is a crystalline solid with a high melting point (152-153° C.) and with a relatively high lipophilicity (log P 3.32). The compound has no readily ionisable groups and consequently has a low aqueous solubility of 0.025 mg/ml over a range of pH values including pH of 7.4. The low aqueous solubility of Tonabersat makes both intravenous (IV) and oral (PO) modes of administration problematic. The poor aqueous solubility prevents rapid injection of the required dose of Tonabersat which is required for the treatment of head injuries and stroke or for emergency treatment of epileptic seizures where the patient may be unconscious and unable to swallow an oral drug. At present the effective plasma concentrations needed to reduce the cortical spreading depression caused by head injury or stroke can only be reached by slow IV infusion given over a period of hours. With respect to the PO administration of Tonabersat for the treatment of other indications, solubility limited dissolution of the tablet form of Tonabersat given PO leads to a significant “food effect” with differences in the maximum blood concentration of Tonabersat (Cmax) seen depending on whether the drug is given with or without food. These differences make it difficult to accurately predict the plasma exposure of Tonabersat when given orally, thus increasing the risk of under or over dosing the patient.

Therefore it is an object of the present invention to provide gap junction blocker compounds having improved physiochemical properties thus improving the utility of these agents in treating a range of disease states.

### BRIEF DESCRIPTION OF THE INVENTION

The present invention makes available three classes of compounds, each class having one or more solubilising pro-drug groups.

### DETAILED DESCRIPTION OF THE INVENTION

In a first aspect, the present invention makes available a class of compounds of formula (Ia) or (Ib), or a hydrate, solvate, or pharmaceutically acceptable salt thereof:

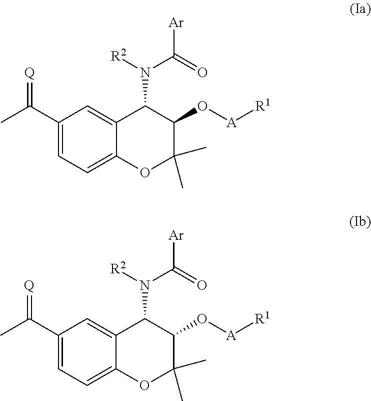

wherein in the compounds of formula (Ia):
Ar is a monocyclic 5 or 6-membered heteroaryl ring or a phenyl ring, either of which is optionally substituted with one or more substituents selected from hydrogen, fluoro, chloro and iodo; and
wherein in the compounds of formula (Ib):
Ar is a monocyclic 5 or 6-membered heteroaryl ring optionally substituted with one or more substituents selected from hydrogen, fluoro, chloro, and iodo; or a phenyl group of formula (IIa):

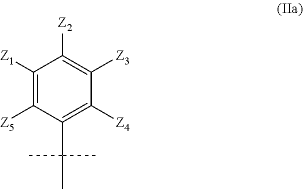

wherein Z₁, Z₂, Z₃, Z₄, and Z₅, are each independently selected from hydrogen fluoro, chloro and iodo, provided that at least one of Z₁ to Z₅ is iodo, and/or at least one of Z₄ and Z₅ is other than hydrogen; and
wherein in the compounds of formula (Ia) and (Ib):
Q is oxygen; R² is hydrogen;

A is a direct bond, —C(O)O*—, —C(O)NH*, —C(R³)(R⁴)O*—, —C(O)O—C(R³)(R⁴)O*—, or —C(R³)(R⁴)O—C(O)O*— wherein the atom marked * is directly connected to R¹,
R³ and R⁴ are selected independently from H, fluoro, C₁₋₄ alkyl, or C₁₋₄ fluoroalkyl, or R³ and R⁴ together with the atom to which they are attached form a cyclopropyl group;
R¹ is selected from groups [1] to [18] wherein the atom marked ** is directly connected to A:

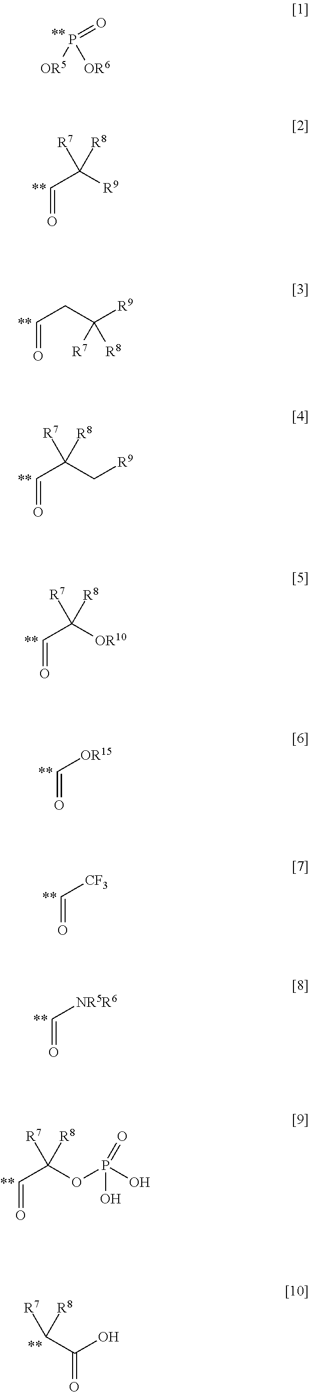

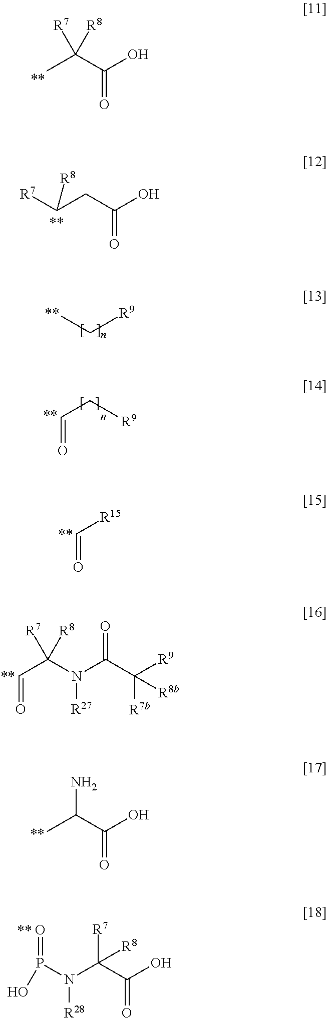

n is 0, 1, 2, or 3,
R⁵ and R⁶ are independently selected from H, C₁₋₄ alkyl, C₁₋₄ fluoroalkyl, —CH₂CH(OH)CH₂OH, or —CH₂CH₂R⁹, or benzyl;
R⁷ and R^{7b} are independently selected from H, C₁₋₄ alkyl, or C₁₋₄ fluoroalkyl;
R⁸ and R^{8b} are independently selected from:
(i) H, C₁₋₄ alkyl, or C₁₋₄ fluoroalkyl, or
(ii) the side chain of a natural or unnatural alpha-amino acid
or R⁷ and R⁸ together with the atom to which they are attached form a C₃₋₇ carbocyclic ring;
R⁹ is selected from hydrogen, —N(R¹¹)(R¹²), or —N⁺(R¹¹)(R¹²)(R¹³)X⁻, —N(R¹¹)C(O)R¹⁴—SO₃H, or —OP(O)(OH)₂,
wherein R¹¹, R¹², and R¹³ are independently selected from hydrogen, C₁₋₄ alkyl, or C₁₋₄ fluoroalkyl, or
R¹¹ and R¹² together with the nitrogen atom to which they are attached form a 3-8 membered heterocyclic ring optionally substituted with one or more substituents selected from hydrogen, fluoro, C₁₋₄ alkyl, C₁₋₄ fluoroalkyl, C₁₋₄ alkoxy, or —C(O)R₃;
or in the case where R¹ is group [16], and R⁹ is —NR¹¹R¹², and R¹¹ is hydrogen, C₁₋₄ alkyl, or C₁₋₄ fluoroalkyl, and R¹² is C₁₋₄ alkyl, or C₁₋₄ fluoroalkyl, then R¹² may join together with R^{8b} such that R¹² and R^{8b} together with the nitrogen to which R¹² is attached form a 5 or 6 membered cyclic amine group,
R¹⁴ is hydrogen, C₁₋₄ alkyl, or C₁₋₄ fluoroalkyl;
R¹⁰ is independently selected from C₁₋₄ alkyl or C₁₋₄ fluoroalkyl, and R¹⁵ is independently selected from C₁₋₄ alkyl or C₁₋₄ fluoroalkyl, 3-pyridyl or 1,4-dihydro-1-methyl-pyridin-3-yl;
R²⁷ is selected from hydrogen, C₁₋₄ alkyl, or C₁₋₄ fluoroalkyl;
R²⁸ is selected from hydrogen, C₁₋₄ alkyl, or C₁₋₄ fluoroalkyl;
X⁻ is a pharmaceutically acceptable anion.

In a second aspect, the present invention makes available a class of compounds of formula (Xa) or (Xb), or a hydrate, solvate, or pharmaceutically acceptable salt thereof:

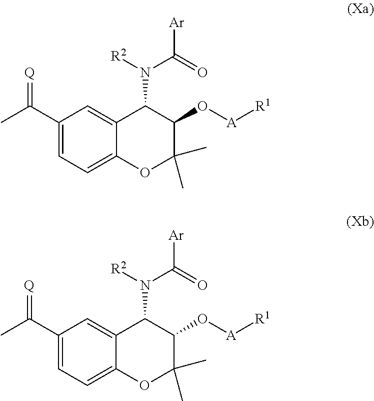

wherein in the compounds of formula (Xa):

Ar is a monocyclic 5 or 6-membered heteroaryl ring or a phenyl ring, either of which is optionally substituted with one or more substituents selected from hydrogen, fluoro, chloro and iodo; and

wherein in the compounds of formula (Xb):

Ar is a monocyclic 5 or 6-membered heteroaryl ring optionally substituted with one or more substituents selected from hydrogen, fluoro, chloro, and iodo; or

a phenyl group of formula (IIa):

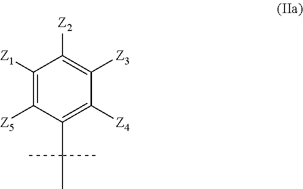

wherein Z₁, Z₂, Z₃, Z₄, and Z₅, are each independently selected from hydrogen fluoro, chloro and iodo, provided that at least one of Z₁ to Z₅ is iodo, and/or at least one of Z₄ and Z₅ is other than hydrogen; and

wherein in the compounds of formula (Xa) and (Xb):

A is a direct bond, and R¹ is H;

Q is O; R² is B—R²¹ wherein,

B is a direct bond, —C(O)O*—, —C(O)NH*, —C(R²³)(R²⁴)O*—, —C(O)O—C(R²³)(R²⁴)O*—, or —C(R²³)(R²⁴)O—C(O)O*— wherein the atom marked * is directly connected to R²¹,

R²³ and R²⁴ are selected independently from H, fluoro, C₁₋₄ alkyl, or C₁₋₄ fluoroalkyl, or R³ and R⁴ together with the atom to which they are attached form a cyclopropyl group;

R²¹ is selected from groups [1] to [18] wherein the atom marked ** is directly connected to B:

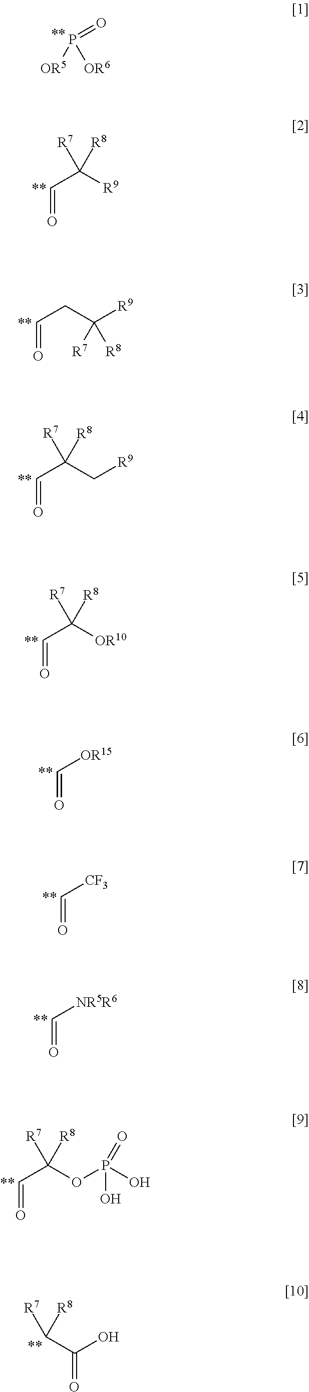

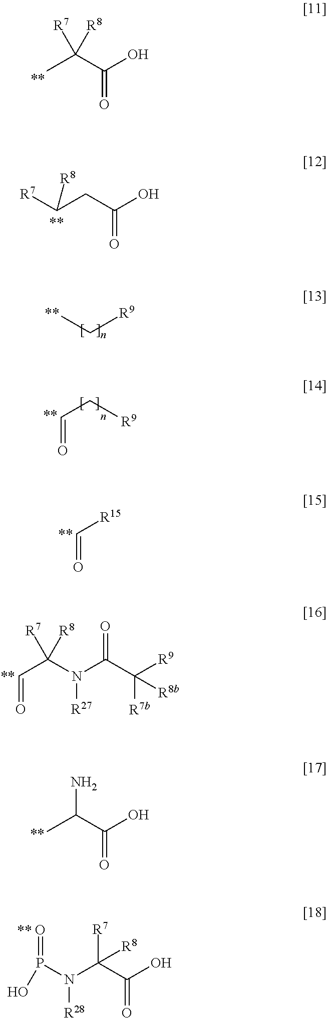

n is 0, 1, 2, or 3,

R⁵ and R⁶ are independently selected from H, C₁₋₄ alkyl, C₁₋₄ fluoroalkyl, —CH₂CH(OH)CH₂OH, or —CH₂CH₂R⁹, or benzyl;

R⁷ and R^{7b} are independently selected from H, C₁₋₄ alkyl, or C₁₋₄ fluoroalkyl;

R⁸ and R^{8b} are independently selected from:
(i) H, C₁₋₄ alkyl, or C₁₋₄ fluoroalkyl, or
(ii) the side chain of a natural or unnatural alpha-amino acid

or R⁷ and R⁸ together with the atom to which they are attached form a C₃₋₇ carbocyclic ring;

R⁹ is selected from hydrogen, —N(R¹¹)(R¹²), or —N⁺(R¹¹)(R¹²)(R¹³)X⁻, —N(R¹¹)C(O)R¹⁴—SO₃H, or —OP(O)(OH)₂,

wherein R¹¹, R¹², and R¹³ are independently selected from hydrogen, C₁₋₄ alkyl, or C₁₋₄ fluoroalkyl, or

R¹¹ and R¹² together with the nitrogen atom to which they are attached form a 3-8 membered heterocyclic ring optionally substituted with one or more substituents selected from hydrogen, fluoro, C₁₋₄ alkyl, C₁₋₄ fluoroalkyl, C₁₋₄ alkoxy, or —C(O)R₃;

or in the case where R¹ is group [16], and R⁹ is —NR¹¹R¹², and R¹¹ is hydrogen, C₁₋₄ alkyl, or C₁₋₄ fluoroalkyl, and R¹² is C₁₋₄ alkyl, or C₁₋₄ fluoroalkyl, then R¹² may join together with R^{8b} such that R¹² and R^{8b} together with the nitrogen to which R¹² is attached form a 5 or 6 membered cyclic amine group,

R¹⁴ is hydrogen, C₁₋₄ alkyl, or C₁₋₄ fluoroalkyl;

R¹⁰ is independently selected from C₁₋₄ alkyl or C₁₋₄ fluoroalkyl, and R¹⁵ is independently selected from C₁₋₄ alkyl or C₁₋₄ fluoroalkyl, 3-pyridyl or 1,4-dihydro-1-methyl-pyridin-3-yl;

R²⁷ is selected from hydrogen, C₁₋₄ alkyl, or C₁₋₄ fluoroalkyl;

R²⁸ is selected from hydrogen, C₁₋₄ alkyl, or C₁₋₄ fluoroalkyl;

X⁻ is a pharmaceutically acceptable anion.

In a third aspect, the present invention makes available a class of compounds of formula (Xla) or (Xlb), or a hydrate, solvate, or pharmaceutically acceptable salt thereof:

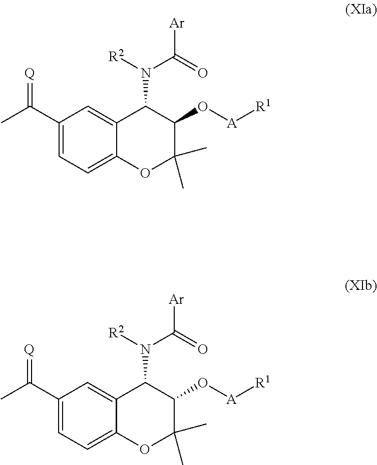

wherein in the compounds of formula (XIa):

Ar is a monocyclic 5 or 6-membered heteroaryl ring or a phenyl ring, either of which is optionally substituted with one or more substituents selected from hydrogen, fluoro, chloro and iodo; and

wherein in the compounds of formula (XIb):

Ar is a monocyclic 5 or 6-membered heteroaryl ring optionally substituted with one or more substituents selected from hydrogen, fluoro, chloro, and iodo; or
a phenyl group of formula (IIa):

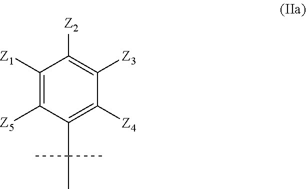

wherein Z₁, Z₂, Z₃, Z₄, and Z₅, are each independently selected from hydrogen fluoro, chloro and iodo, provided that at least one of Z₁ to Z₅ is iodo, and/or at least one of Z₄ and Z₅ is other than hydrogen; and

wherein in the compounds of formula (XIa) and (XIb):

R² and -A-R¹ are both H;

Q is an acetal or hemiacetal group of formula —C(OR⁴¹)(OR⁴²)— wherein

R⁴¹ and R⁴² are independently H, C₁₋₄ fluoroalkyl or optionally substituted C₁₋₄ alkyl, or R⁴¹ and R⁴² together with the carbon atom to which they are attached form a 5-8 membered heterocycle, any carbon atom of which is optionally substituted; or

Q is an oxime of formula ═NHOR⁴³, wherein R⁴³ is
(i) selected from H, C₁₋₄ fluoroalkyl or optionally substituted C₁₋₄ alkyl, or
(ii) -A-R¹ wherein A and R¹ are as set out in claim 1
X⁻ is a pharmaceutically acceptable anion.

In an embodiment R⁴³ is C₁₋₄ alkyl optionally substituted with a phosphate group (—P(O)OR⁶¹OR⁶²). In an example of such an embodiment OR⁴³ is —OCH₂P(O)OR⁶¹OR⁶², wherein R⁶¹ and R⁶² are independently H or C₁₋₄ alkyl.

In another embodiment R⁴³ is an amino acid derivative having the structure —C(O)CH(R¹⁰⁰)NH₂ wherein the group R¹⁰⁰ is the side chain of a natural or unnatural amino acid. In an embodiment OR⁴³ is —OC(O)CH(CH(CH₃)₂)NH₂.

Terminology

As used herein, the term “includes” means including the following integers, but not limited thereto.

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein, the term “(Cₐ-C_{b})fluoroalkyl” has the same meaning as “(Cₐ-C_{b})alkyl” except that one or more of the hydrogen atoms directly connected to the carbon atoms forming the alkyl group is replaced by the corresponding number of fluorine atoms.

As used herein the unqualified term “carbocyclic” refers to a mono-, bi- or tricyclic radical having up to 16 ring atoms, all of which are carbon, and includes aryl and cycloalkyl.

As used herein the unqualified term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, triazinyl, indolyl and indazolyl. In an embodiment of the present invention, heteroaryl is oxadiazolyl, pyridinyl, pyridazinyl, pyrimidinyl, indolyl and indazolyl.

As used herein the unqualified term “heterocyclyl” or “heterocyclic” includes “heteroaryl” as defined above, and in addition means a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

When the term cyclic amino group is used the cyclic amino groups can have 3-8 ring atoms, 3-7 ring atoms, 5-7 ring atoms, 5-6 ring atoms. When the terms 3-8 or 3-7 cyclic amino group is used all ranges within those ranges are disclosed, for example 3-8 includes 3-7. Both 3-8 and 3-7 include 4-7 and 5-7 and 5-6. Examples of 5 and 6 membered cyclic amino groups include morpholine, piperidine, piperazine, pyrrolidine.

Unless otherwise specified in the context in which it occurs, the term “substituted” as applied to any moiety herein means substituted with up to four compatible substituents, each of which independently may be, for example, (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, halo (including fluoro, bromo and chloro), fully or partially fluorinated (C₁-C₃)alkyl, (C₁-C₃)alkoxy or (C₁-C₃)alkylthio such as trifluoromethyl, trifluoromethoxy, and trifluoromethylthio, nitro, nitrile (—CN), oxo, phenyl, phenoxy, monocyclic heteroaryl or heteroaryloxy with 5 or 6 ring atoms, tetrazolyl, —COOR^{A}, —COR^{A}, —OCOR^{A}, —SO₂R^{A}, —CONR^{A}R^{B}, —SO₂NR^{A}R^{B}, —NR^{A}R^{B}, OCONR^{A}R^{B}, —NR^{B}COR^{A}, —NR^{B}COOR^{A}, —NR^{B}SO₂OR^{A} or —NR^{A}CONR^{A}R^{B} wherein R^{A} and R^{B} are independently hydrogen or a (C₁-C₆)alkyl group or, in the case where R^{A} and R^{B} are linked to the same N atom, R^{A} and R^{B} taken together with that nitrogen may form a cyclic amino ring, such as a morpholine, piperidinyl or piperazinyl ring. Where the substituent is phenyl, phenoxy or monocyclic heteroaryl or heteroaryloxy with 5 or 6 ring atoms, the phenyl or heteroaryl ring thereof may itself be substituted by any of the above substituents except phenyl, phenoxy, heteroaryl or heteroaryloxy. An “optional substituent” may be one of the foregoing substituent groups.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Compounds of the inventions which are basic can form salts, including pharmaceutically acceptable salts with inorganic acids, e.g. hydrohalic acids such as hydrochloric or hydrobromic acids, sulphuric acid, nitric acid or phosphoric acid and the like, and with organic acids e.g. acetic, tartaric, succinic, fumaric, maleic, malic, salicylic, citric, methanesulphonic, p-toluenesulphonic, benzoic, benzenesunfonic, glutamic, lactic, and mandelic acids and the like.

The formation of specific salt forms can provide compounds of the invention with improved physicochemical properties. For a review on suitable salts, see Handbook of Pharmaceutical Salts: Properties, Selection, and Use by Stahl and Wermuth (Wiley-VCH, Weinheim, Germany, 2002).

The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Compounds with which the invention is concerned which may exist in one or more stereoisomeric form, because of the presence of asymmetric atoms or rotational restrictions, can exist as a number of stereoisomers with R or S stereochemistry at each chiral centre or as atropisomers with R or S stereochemistry at each chiral axis. The invention includes all such enantiomers and diastereoisomers and mixtures thereof. In particular the carbon atom to which the R⁸ or R^{8b} substituent is attached may be in either the R or the S stereochemical configuration.

The compounds of the invention include compounds of formula (Ia), (Ib), (Xa), (Xb) and (XIa) and (XIb) as hereinbefore defined, including all polymorphs and crystal habits thereof, and isomers thereof (including optical, geometric and tautomeric isomers) as hereinafter defined and isotopically-labeled compounds of formula (Ia), (Ib), (Xa), (Xb) and (XIa) and (XIb).

Examples of side chains of natural alpha amino acids include those of alanine, arginine, asparagine, aspartic acid, cysteine, cystine, glutamic acid, histidine, 5-hydroxylysine, 4-hydroxyproline, isoleucine, leucine, lysine, methionine, phenylalanine, proline, serine, threonine, tryptophan, tyrosine, valine, -aminoadipic acid, α-amino-n-butyric acid, 3,4-dihydroxyphenylalanine, homoserine, α-methylserine, ornithine, pipecolic acid, and thyroxine.

Natural alpha-amino acids which contain functional substituents, for example amino, carboxyl, hydroxy, mercapto, guanidyl, imidazolyl, or indolyl groups in their characteristic side chains include arginine, lysine, glutamic acid, aspartic acid, tryptophan, histidine, serine, threonine, tyrosine, and cysteine. When R⁸ or R^{8b} in the compounds of the invention is one of those side chains, the functional substituent may optionally be protected.

The term “protected” when used in relation to a functional substituent in a side chain of a natural alpha-amino acid means a derivative of such a substituent which is substantially non-functional. For example, carboxyl groups may be esterified (for example as a C₁-C₆ alkyl ester), amino groups may be converted to amides (for example as a NHCOC₁-C₆ alkyl amide) or carbamates (for example as an NHC(═O)OC₁-C₆ alkyl or NHC(═O)OCH₂Ph carbamate), hydroxyl groups may be converted to ethers (for example an OC₁-C₆ alkyl or a O(C₁-C₆ alkyl)phenyl ether) or esters (for example a OC(═O)C₁-C₆ alkyl ester) and thiol groups may be converted to thioethers (for example a tert-butyl or benzyl thioether) or thioesters (for example a SC(═O)C₁-C₆ alkyl thioester).

Examples of side chains of non-natural alpha amino acids include:

an optional substituent, C₁-C₆ alkyl, phenyl, 2,- 3-, or 4-hydroxyphenyl, 2,- 3-, or 4-methoxyphenyl, 2,-3-, or 4-pyridylmethyl, benzyl, phenylethyl, 2-, 3-, or 4-hydroxybenzyl, 2,- 3-, or 4-benzyloxybenzyl, 2,- 3-, or 4-C₁-C₆ alkoxybenzyl, and benzyloxy(C₁-C₆alkyl)-groups, wherein any of the foregoing non-natural amino acid side chains is optionally substituted in the alkyl, phenyl or pyridyl group; or groups -[Alk]ₙR₅₀ where Alk is a (C₁-C₆)alkyl or (C₂-C₆)alkenyl group optionally interrupted by one or more —O—, or —S— atoms or —N(R₅₁)— groups [where R₅₁ is a hydrogen atom or a (C₁-C₆)alkyl group], n is 0 or 1, and R₅₀ is an optionally substituted cycloalkyl or cycloalkenyl group; or

a heterocyclic(C₁-C₆)alkyl group, either being unsubstituted or mono- or di-substituted in the heterocyclic ring with halo, nitro, carboxy, (C₁-C₆)alkoxy, cyano, (C₁-C₆)alkanoyl, trifluoromethyl (C₁-C₆)alkyl, hydroxy, formyl, amino, (C₁-C₆)alkylamino, di-(C₁-C₆)alkylamino, mercapto, (C₁-C₆)alkylthio, hydroxy(C₁-C₆)alkyl, mercapto(C₁-C₆)alkyl or (C₁-C₆)alkylphenylmethyl.

The Group Ar is as set out in claim 1. In an embodiment, the compounds of formula (Ia) and (Ib) have an Ar group which is an optionally substituted thiophenyl ring, the optional substituents being fluoro, chloro, iodo or hydrogen (H).

In an embodiment, the compounds of formula (Ib) have an Ar group selected from the group consisting of:

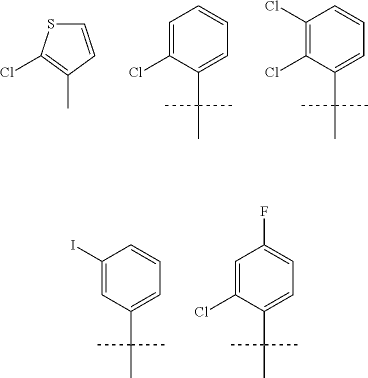

In an embodiment, the compounds of formula (Ia) have an Ar selected from the group consisting of:

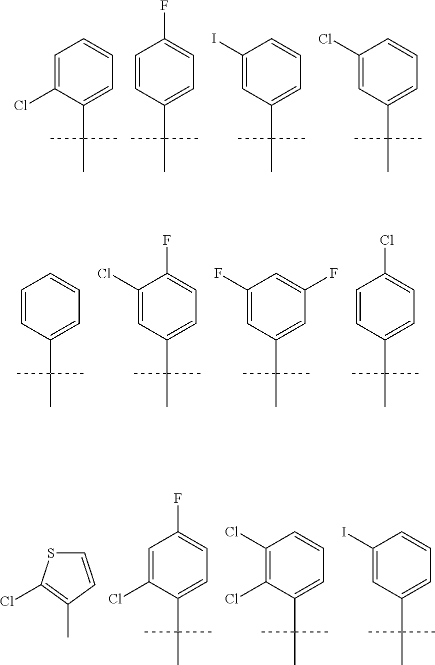

The Group A is as set out in claim 1.

A is a direct bond, —C(O)O*—, —C(O)NH*, —C(R³)(R⁴)O*—, —C(O)O—C(R³)(R⁴)O*—, or —C(R³)(R⁴)O—C(O)O*— wherein the atom marked * is directly connected to R¹. In an embodiment, A is a direct bond, or —C(R³)(R⁴)O*—

R³ and R⁴ are selected independently from H, fluoro, C₁₋₄ alkyl such as methyl, ethyl or isopropyl, or C₁₋₄ fluoroalkyl such as trifluoromethyl, or R³ and R⁴ together with the atom to which they are attached form a cyclopropyl group. In an embodiment R³ and R⁴ are both hydrogen, or R³ and R⁴ are both C₁₋₄ alkyl, or R³ is H and R⁴ is C₁₋₄ alkyl.

The group R¹ is as set out in claim 1.

In an embodiment, A is a direct bond or —C(R³)(R⁴)O*—, and R¹ is:

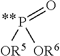

wherein R⁵ and R⁶ are independently selected from hydrogen and C₁₋₄ alkyl; and R³ and R⁴ are selected independently from H, fluoro, C₁₋₄ alkyl such as methyl, ethyl or isopropyl, or C₁₋₄ fluoroalkyl such as trifluoromethyl. In an embodiment, R³ and R⁴ are both hydrogen. In an embodiment, R⁵ and R⁶ are both hydrogen.

In an embodiment, the compounds of the invention have the formula (Ya) and (Za), wherein Ar is as defined in claim 1 for formula (Ia):

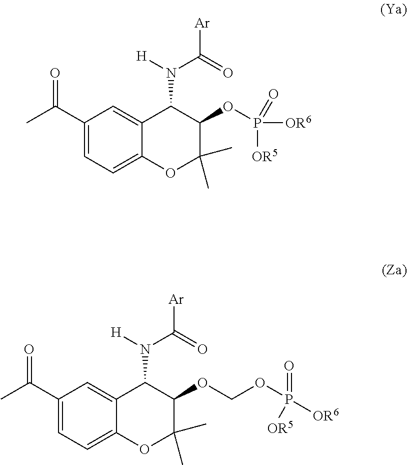

wherein R⁵ and R⁶ are independently selected from hydrogen and C₁₋₄ alkyl.

In an embodiment, the compounds of the invention have the formula (Yb) and (Zb), wherein Ar is as defined in claim 1 for formula (Ib):

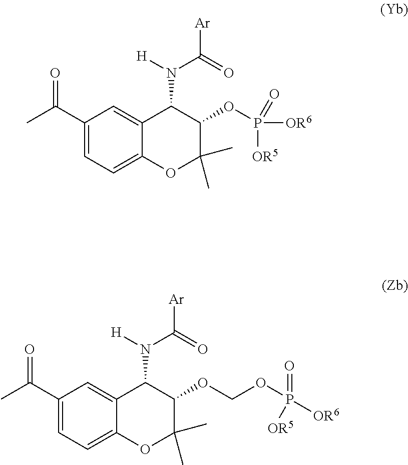

wherein R⁵ and R⁶ are independently selected from hydrogen and C₁₋₄ alkyl.

In an embodiment, the compounds of the invention have the formula (YIa) wherein Ar is as defined in claim 1 for formula (Ia):

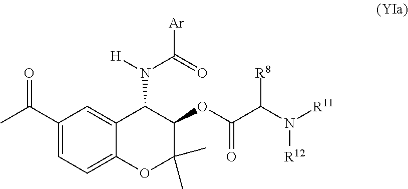

wherein R⁸ is the side chain of a natural alpha amino acid, and wherein R¹¹, R¹² are independently selected from hydrogen, C₁₋₄ alkyl, or C₁₋₄ fluoroalkyl. In an embodiment, the side chain of the natural alpha-amino acid is selected from hydrogen, —CH(CH₃)₂, —(CH₂)₃CH₂NH₂, —CH(CH₃)(CH₂CH₂CH₃), —CH₂CH(CH₃)₂, —CH₂OH, or the histidine side chain:

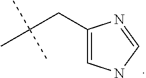

In an embodiment, R¹¹ is hydrogen, and R¹² is C₁₋₄ alkyl selected from methyl, ethyl, propyl and isopropyl. In an embodiment, the carbon atom bearing the R⁸ group has the natural L-amino acid stereochemistry.

In an embodiment the compounds of the invention have the formula (YIb) wherein Ar is as defined in claim 1 for formula (Ib):

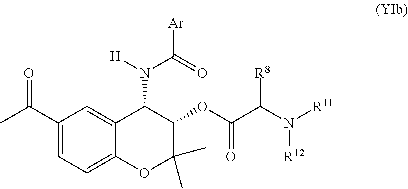

wherein R⁸ is the side chain of a natural alpha-amino acid, and wherein R¹¹, R¹² are independently selected from hydrogen, C₁₋₄ alkyl, or C₁₋₄ fluoroalkyl. In an embodiment, the side chain of the natural amino acid is selected from hydrogen, —CH(CH₃)₂, —(CH₂)₃CH₂NH₂, —CH(CH₃)(CH₂CH₂CH₃), —CH₂CH(CH₃)₂, —CH₂OH, or the histidine side chain:

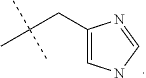

In an embodiment, R¹¹ is hydrogen, and R¹² is C₁₋₄ alkyl selected from methyl, ethyl, propyl and isopropyl. In an embodiment, the carbon atom bearing the R⁸ group has the natural L-amino acid stereochemistry.

In an embodiment, the compounds of the invention have the formula (YIIa) wherein Ar is as defined in claim 1 for formula (Ia):

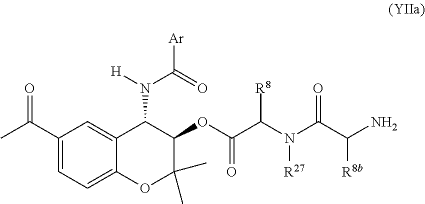

wherein R²⁷ is hydrogen or C₁₋₄ alkyl; and R⁸ and R^{8b} are each independently the side chain of a natural alpha-amino acid. In an embodiment, the side chain of the natural amino acid is selected from hydrogen, hydrogen, CH₃, —CH(CH₃)₂, —(CH₂)₃CH₂NH₂, —CH(CH₃)(CH₂CH₂CH₃), —CH₂CH(CH₃)₂, —CH₂OH, or the histidine side chain:

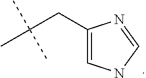

In an embodiment R²⁷ is hydrogen or methyl. In an embodiment, the carbon atoms bearing the R⁸ and R^{8b} groups have the natural L-amino acid stereochemistry.

In an embodiment, the compounds of the invention have the formula (YIIb) wherein Ar is as defined in claim 1 for formula (Ib):

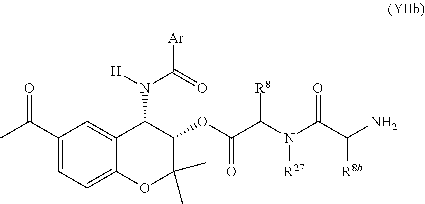

wherein R²⁷ is hydrogen or C₁₋₄ alkyl; and R⁸ and R^{8b} are each independently the side chain of a natural alpha-amino acid. In an embodiment, the side chain of the natural amino acid is selected from hydrogen, hydrogen, CH₃, —CH(CH₃)₂, —(CH₂)₃CH₂NH₂, —CH(CH₃)(CH₂CH₂CH₃), —CH₂CH(CH₃)₂, —CH₂OH, or the histidine side chain:

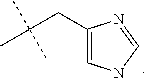

In an embodiment R²⁷ is hydrogen or methyl. In an embodiment, the carbon atoms bearing the R⁸ and R8b groups have the natural L-amino acid stereochemistry.

In an embodiment, the compounds of the invention have the formula (YIIIa) wherein Ar is as defined in claim 1 for formula (Ia):

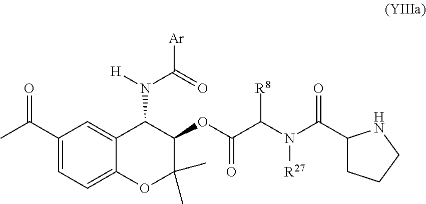

Wherein R⁸ is the side chain of a natural alpha-amino acid, and R²⁷ is hydrogen or C₁₋₄ alkyl. In an embodiment the right hand group is the natural amino acid proline.

In an embodiment, the compounds of the invention have the formula (YIIIb) wherein Ar is as defined in claim 1 for formula (Ib):

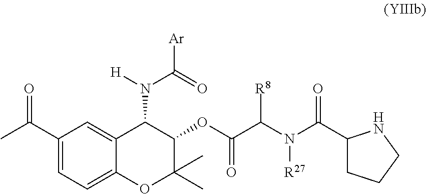

Wherein R⁸ is the side chain of a natural alpha-amino acid, and R²⁷ is hydrogen or C₁₋₄ alkyl. In an embodiment the right hand group is the natural amino acid proline.

In an embodiment of the compounds set out in claim 1, A is a direct bond and R¹ has the formula (7A):

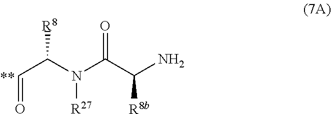

wherein R²⁷ is hydrogen or C₁₋₄ alkyl; and R⁸ and R^{8b} are each independently the side chain of a natural alpha-amino acid.

In an embodiment, R¹¹, R¹², and R¹³ are independently methyl or ethyl.

In an embodiment, R¹¹ and R¹² together with the nitrogen atom to which they are attached form a 5, 6 or 7 membered cyclic amino group such as morpholine, pyrrolidine, piperidine, piperazine, homopiperidine, and homopiperazine.

In an embodiment, R⁷ is hydrogen and R⁸ is the side chain of a natural or unnatural amino acid.

In an embodiment, R^{7b} is hydrogen and R^{8b} is the side chain of a natural or unnatural amino acid.

In an embodiment, the side chain of the natural or unnatural amino acid is selected from —CH(CH₃)₂, —(CH₂)₃CH₂NH₂, —CH(CH₃)(CH₂CH₂CH₃), —CH₂CH(CH₃)₂, —CH₂OH, or the histidine side chain:

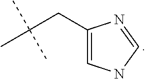

In an embodiment, R⁷ and R⁸ are both hydrogen.

In an embodiment, R^{7b} and R^{8b} are both hydrogen.

In an embodiment, R⁶ is selected from —CH₂CH(OH)CH₂OH, —CH₂CH₂NR¹¹R¹², or —CH₂CH₂NR¹¹R¹²R¹³X⁻.

In an embodiment, R⁵ and R⁶ are hydrogen.

In an embodiment, the group A is as defied in claim 1, and R¹ is selected from any one of the following groups:

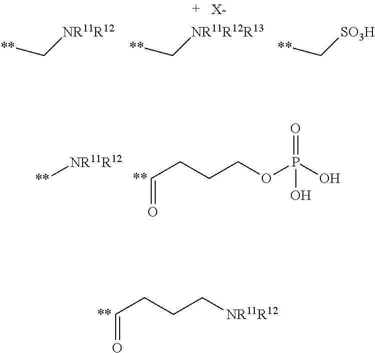

In an embodiment the group -A-R¹ is selected from the following groups, wherein the atom marked ** is directly connected to the oxygen atom:

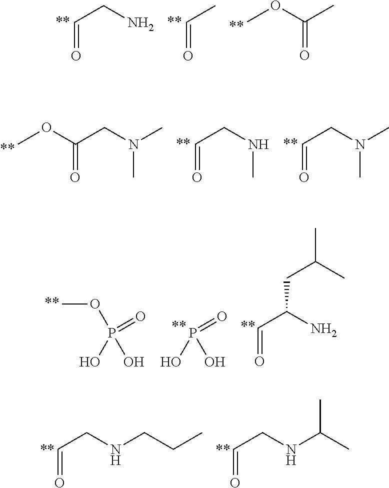

It will be understood that the compounds of formula (Ia), (Ib), (Xa), (Xb), (XIa), and (XIb) may be further modified by adding one or more prodrug groups such as those defined by Q, -AR¹ and R². For example the compounds of formula (Ia) or (Ib) may be modified by exchanging the oxygen atom Q with a prodrug group, such as Q as defined in (XIa) or (XIb). Alternatively, the compounds of formula (Ia) or (Ib) can be modified by replacing the hydrogen atom R² by a prodrug group such as R² as defined in formula (Xa) and (Xb).

In an embodiment, the compounds of formula (Ia) and (Ib) are selected from the Examples, and pharmaceutically acceptable salts thereof.

In an embodiment, the compounds of formula (Ia) and (Ib) are selected from the following compounds:

{[(3R,4S)-6-Acetyl-4-[(4-fluorobenzene)amido]-2,2-dimethyl-3,4-dihydro-2H-1-benzopyran-3-yl]oxy}phosphonic acid

({[(3R,4S)-6-Acetyl-4-[(4-fluorobenzene)amido]-2,2-dimethyl-3,4-dihydro-2H-1-benzopyran-3-yl]oxy}methoxy)phosphonic acid

(3R,4S)-6-Acetyl-4-[(4-fluorobenzene)amido]-2,2-dimethyl-3,4-dihydro-2H-1-benzopyran-3-yl (2S)-2-amino-3-methylbutanoate hydrochloride

(3R,4S)-6-Acetyl-4-[(4-fluorobenzene)amido]-2,2-dimethyl-3,4-dihydro-2H-1-benzopyran-3-yl (2S)-2-[(2S)-2-amino-3-methyl butanamido]-3-methylbutanoate

(3R,4S)-6-Acetyl-4-[(4-fluorobenzene)amido]-2,2-dimethyl-3,4-dihydro-2H-1-benzopyran-3-yl 2-[(2S)-pyrrolidin-2-ylformamido]acetate

(3R,4S)-6-Acetyl-4-[(4-fluorobenzene)amido]-2,2-dimethyl-3,4-dihydro-2H-1-benzopyran-3-yl 2-[(propan-2-yl)amino]acetate

(3R,4S)-6-Acetyl-4-[(4-fluorobenzene)amido]-2,2-dimethyl-3,4-dihydro-2H-1-benzopyran-3-yl 2-(morpholin-4-yl)ethyl carbonate

{[(3S,4S)-6-Acetyl-4-[(2-chlorobenzene)amido]-2,2-dimethyl-3,4-dihydro-2H-1-benzopyran-3-yl]oxy}phosphonic acid

{[(3S,4S)-6-Acetyl-4-[(2,3-dichlorobenzene)amido]-2,2-dimethyl-3,4-dihydro-2H-1-benzopyran-3-yl]oxy}phosphonic acid

{[(3S,4S)-6-Acetyl-4-(2-chlorothiophene-3-amido)-2,2-dimethyl-3,4-dihydro-2H-1-benzopyran-3-yl]oxy}phosphonic acid

{[(3R,4S)-6-Acetyl-4-[(3-iodobenzene)amido]-2,2-dimethyl-3,4-dihydro-2H-1-benzopyran-3-yl]oxy}phosphonic acid

{[(3R,4S)-6-Acetyl-4-[(3-chlorobenzene)amido]-2,2-dimethyl-3,4-dihydro-2H-1-benzopyran-3-yl]oxy}phosphonic acid

{[(3R,4S)-6-Acetyl-4-[(2,3-dichlorobenzene)amido]-2,2-dimethyl-3,4-dihydro-2H-1-benzopyran-3-yl]oxy}phosphonic acid

{[(3R,4S)-6-Acetyl-4-[(3-chloro-4-fluorobenzene)amido]-2,2-dimethyl-3,4-dihydro-2H-1-benzopyran-3-yl]oxy}phosphonic acid

{[(3R,4S)-6-Acetyl-4-(2-chlorothiophene-3-amido)-2,2-dimethyl-3,4-dihydro-2H-1-benzopyran-3-yl]oxy}phosphonic acid

{[(3R,4S)-6-Acetyl-4-[(2-chloro-4-fluorobenzene)amido]-2,2-dimethyl-3,4-dihydro-2H-1-benzopyran-3-yl]oxy}phosphonic acid

{[(3R,4S)-6-Acetyl-4-[(2-chlorobenzene)amido]-2,2-dimethyl-3,4-dihydro-2H-1-benzopyran-3-yl]oxy}phosphonic acid

{[(3R,4S)-6-Acetyl-4-benzamido-2,2-dimethyl-3,4-dihydro-2H-1-benzopyran-3-yl]oxy}phosphonic acid

{[(3R,4S)-6-Acetyl-4-[(4-chlorobenzene)amido]-2,2-dimethyl-3,4-dihydro-2H-1-benzopyran-3-yl]oxy}phosphonic acid

{[(3R,4S)-6-Acetyl-4-[(3,5-difluorobenzene)amido]-2,2-dimethyl-3,4-dihydro-2H-1-benzopyran-3-yl]oxy}phosphonic acid

({[(3S,4S)-6-Acetyl-4-[(2-chlorobenzene)amido]-2,2-dimethyl-3,4-dihydro-2H-1-benzopyran-3-yl]oxy}methoxy)phosphonic acid

({[(3S,4S)-6-Acetyl-4-[(2,3-dichlorobenzene)amido]-2,2-dimethyl-3,4-dihydro-2H-1-benzopyran-3-yl]oxy}methoxy)phosphonic acid

({[(3S,4S)-6-Acetyl-4-(2-chlorothiophene-3-amido)-2,2-dimethyl-3,4-dihydro-2H-1-benzopyran-3-yl]oxy}methoxy)phosphonic acid

({[(3R,4S)-6-Acetyl-4-[(3-iodobenzene)amido]-2,2-dimethyl-3,4-dihydro-2H-1-benzopyran-3-yl]oxy}methoxy)phosphonic acid

({[(3R,4S)-6-Acetyl-4-[(3-chlorobenzene)amido]-2,2-dimethyl-3,4-dihydro-2H-1-benzopyran-3-yl]oxy}methoxy)phosphonic acid

({[(3R,4S)-6-Acetyl-4-[(2,3-dichlorobenzene)amido]-2,2-dimethyl-3,4-dihydro-2H-1-benzopyran-3-yl]oxy}methoxy)phosphonic acid

({[(3R,4S)-6-Acetyl-4-[(3-chloro-4-fluorobenzene)amido]-2,2-dimethyl-3,4-dihydro-2H-1-benzopyran-3-yl]oxy}methoxy)phosphonic acid

({[(3R,4S)-6-Acetyl-4-(2-chlorothiophene-3-amido)-2,2-dimethyl-3,4-dihydro-2H-1-benzopyran-3-yl]oxy}methoxy)phosphonic acid

({[(3R,4S)-6-Acetyl-4-[(2-chloro-4-fluorobenzene)amido]-2,2-dimethyl-3,4-dihydro-2H-1-benzopyran-3-yl]oxy}methoxy)phosphonic acid

({[(3R,4S)-6-Acetyl-4-[(2-chlorobenzene)amido]-2,2-dimethyl-3,4-dihydro-2H-1-benzopyran-3-yl]oxy}methoxy)phosphonic acid

({[(3R,4S)-6-Acetyl-4-benzamido-2,2-dimethyl-3,4-dihydro-2H-1-benzopyran-3-yl]oxy}methoxy)phosphonic acid

({[(3R,4S)-6-Acetyl-4-[(4-chlorobenzene)amido]-2,2-dimethyl-3,4-dihydro-2H-1-benzopyran-3-yl]oxy}methoxy)phosphonic acid

({[(3R,4S)-6-Acetyl-4-[(3,5-difluorobenzene)amido]-2,2-dimethyl-3,4-dihydro-2H-1-benzopyran-3-yl]oxy}methoxy)phosphonic acid

(3R,4S)-6-Acetyl-4-[(3-chlorobenzene)amido]-2,2-dimethyl-3,4-dihydro-2H-1-benzopyran-3-yl (2S)-2-amino-3-methylbutanoate

(3R,4S)-6-Acetyl-4-[(3-chlorobenzene)amido]-2,2-dimethyl-3,4-dihydro-2H-1-benzopyran-3-yl (2S)-2-[(2S)-2-amino-3-methyl butanamido]-3-methylbutanoate

(3R,4S)-6-Acetyl-4-[(3-chlorobenzene)amido]-2,2-dimethyl-3,4-dihydro-2H-1-benzopyran-3-yl (2S)-2-amino-4-methylpentanoate

(3R,4S)-6-Acetyl-4-[(3-chlorobenzene)amido]-2,2-dimethyl-3,4-dihydro-2H-1-benzopyran-3-yl (2R)-2-amino-4-methylpentanoate

(3R,4S)-6-Acetyl-4-[(3-chlorobenzene)amido]-2,2-dimethyl-3,4-dihydro-2H-1-benzopyran-3-yl 2-[(propan-2-yl)amino]acetate

(3R,4S)-6-Acetyl-4-[(3-chlorobenzene)amido]-2,2-dimethyl-3,4-dihydro-2H-1-benzopyran-3-yl (2S)-2-aminopropanoate

(3R,4S)-6-Acetyl-4-[(3-chlorobenzene)amido]-2,2-dimethyl-3,4-dihydro-2H-1-benzopyran-3-yl (2S)-2-(methylamino)propanoate

(3R,4S)-6-Acetyl-4-[(3-chlorobenzene)amido]-2,2-dimethyl-3,4-dihydro-2H-1-benzopyran-3-yl (2S)-2-[(2S)-2-amino-3-methylbutanamido]propanoate

(3R,4S)-6-Acetyl-4-[(3-chlorobenzene)amido]-2,2-dimethyl-3,4-dihydro-2H-1-benzopyran-3-yl (2S)-2-[(2S)-2-amino-N,3-dimethylbutanamido]propanoate

(3R,4S)-6-Acetyl-4-[(3-chlorobenzene)amido]-2,2-dimethyl-3,4-dihydro-2H-1-benzopyran-3-yl (2S)-2-[(2S)-2-amino-4-methylpentanamido]propanoate

and pharmaceutically acceptable salts thereof.

The present invention makes available a pharmaceutical composition comprising a compound of formula (Ia), (Ib), (Xa), (Xb), (XIa), and (XIb) together with one or more pharmaceutically acceptable carriers and/or excipients. Preferably, the pharmaceutical composition comprises a compound of formula (Ia) or (Ib).

The present invention makes available a compound of formula (Ia), (Ib), (Xa), (Xb), (XIa), and (XIb) for use in medicine. Preferably a compound of formula (Ia) or (Ib).

In an embodiment the inventions encompasses the use of a compound of formula (Ia), (Ib), (Xa), (Xb), (XIa), and (XIb) for treatment of a disease or medical condition which benefits from inhibition of gap junction activity, preferably a compound of formula (Ia) or (Ib). Inhibition of gap junction activity may be achieved by blocking the gap junction as a whole or by blocking one or more hemichannels.

It will be understood that the pharmacology of the brain is a complex and constantly evolving area of research. Without wishing to be bound by theory, it is currently hypothesised that the claimed compounds exert their therapeutic effect by inhibiting gap junction activity. However, it is anticipated that compounds of formula (Ia), (Ib), (Xa), (Xb), (XIa), and (XIb), preferably of formula (Ia) or (Ib), may exert their therapeutic effect by additional and/or alternative mechanisms of action. For the avoidance of doubt, the claimed compounds are expected to be useful for treatment of any one of the diseases selected from among migraine, aura with or without migraine, epilepsy, non-epileptic seizures, cerebrovascular accidents including stroke, intracranial haemorrhage (including or traumatic brain injury, epidural hematoma, subdural hematoma and subarachnoid haemorrhage), and intra-cerebral haemorrhage (including CADASIL), spinal cord vascular accidents arising from trauma, epidural hematoma, subdural hematoma or subarachnoid haemorrhage, pain including pain arising from hyperalgesia caused by damage to sensory neurons (i.e. neuropathic pain including but not limited to diabetic neuropathy, polyneuropathy, cancer pain, fibromyalgia, myofascial pain, post herpetic neuralgia, spinal stenosis, HIV pain, post-operative pain, post-trauma pain) or inflammation (including pain associated with osteoarthritis, rheumatoid arthritis, sciatica/radiculopathy, pancreatitis, tendonitis), neurodegenerative disease (including but not limited to Alzheimer's Disease, Parkinson's Disease, Huntington's Disease and Amyotrophic Lateral Sclerosis) and cardiovascular disease including myocardial infarction, coronary revascularization or angina.

In an embodiment the invention encompasses a method of treatment of a disease or medical condition, comprising administering to a subject suffering from such disease or condition and effective amount of a compound of formula (Ia), (Ib), (Xa), (Xb), (XIa), and (XIb), preferably of formula (Ia) or (Ib), wherein the disease or condition is selected from among migraine, aura with or without migraine, epilepsy, non-epileptic seizures, cerebrovascular accidents including stroke, intracranial haemorrhage (including or traumatic brain injury, epidural hematoma, subdural hematoma and subarachnoid haemorrhage), and intra-cerebral haemorrhage (including CADASIL), spinal cord vascular accidents arising from trauma, epidural hematoma, subdural hematoma or subarachnoid haemorrhage, pain including pain arising from hyperalgesia caused by damage to sensory neurons (i.e. neuropathic pain including but not limited to diabetic neuropathy, polyneuropathy, cancer pain, fibromyalgia, myofascial pain, post herpetic neuralgia, spinal stenosis, HIV pain, post-operative pain, post-trauma pain) or inflammation (including pain associated with osteoarthritis, rheumatoid arthritis, sciatica/radiculopathy, pancreatitis, tendonitis), neurodegenerative disease (including but not limited to Alzheimer's Disease, Parkinson's Disease, Huntington's Disease and Amyotrophic Lateral Sclerosis) and cardiovascular disease including myocardial infarction, coronary revascularization or angina.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, as is required in the pharmaceutical art. However, for administration to human patients, the total daily dose of the compounds of the invention may typically be in the range 1 mg to 1000 mg depending, of course, on the mode of administration. For example, oral administration may require a total daily dose of from 10 mg to 1000 mg, while an intravenous dose may only require from 1 mg to 500 mg. The total daily dose may be administered in single or divided doses and may, at the physician's discretion, fall outside of the typical range given herein. These dosages are based on an average human subject having a weight of about 60 kg to 100 kg. The physician will readily be able to determine doses for subjects whose weight falls outside this range, such as infants and the elderly, and especially obese patients.

The compounds with which the invention is concerned may be prepared for administration by any route consistent with their pharmacokinetic properties. Suitable routes for administration include oral, intravenous, buccal, intranasal, inhalation, rectal, and intradermal. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

The pro-drug may also be administered parenterally in a sterile medium. Depending on the vehicle and concentration used, the drug can either be suspended or dissolved in the vehicle. Advantageously, adjuvants such as local anaesthetic, preservative and buffering agents can be dissolved in the vehicle. The person skilled in the art is aware of many excipients useful for IV formulation.

In an embodiment, the claimed compounds, or a pharmaceutical composition or preparation comprising the claimed compounds is formulated as a liquid for intravenous dosage, or formulated as a solid for oral dosage.

Preparation of Compounds of the Invention

The compounds of formula (Ia) and (Ib) above may be prepared by, or in analogy with, conventional methods. The preparation of intermediates and compounds according to the Examples of the present invention may in particular be illuminated by the following Schemes. Definitions of variables in the structures in Schemes herein are commensurate with those of corresponding positions in the formulas delineated herein.

Scheme 1. General synthetic route for preparation of compounds of formula (Ia) and (Ib)

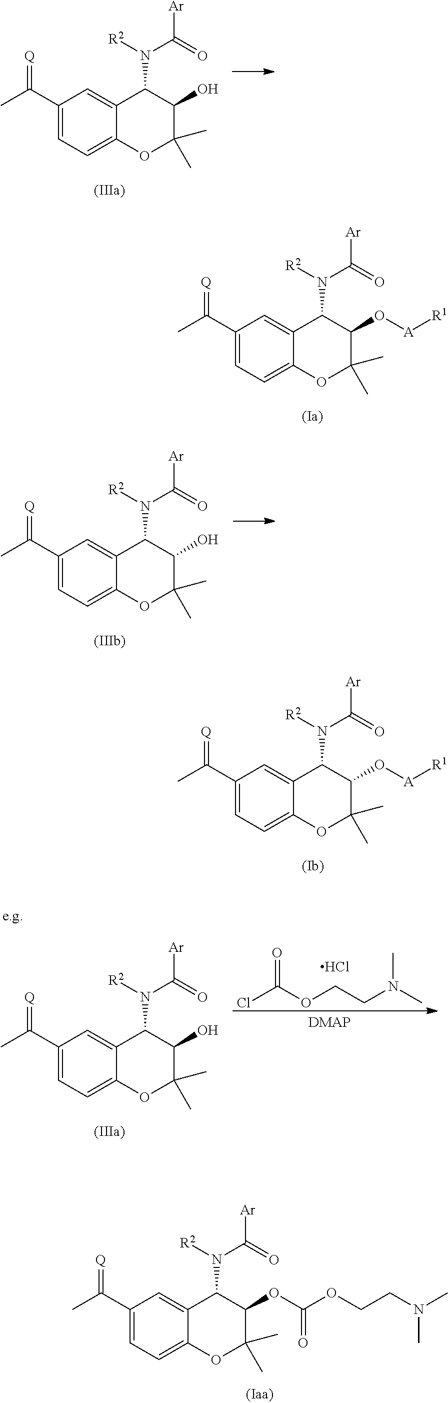

wherein A, Q, Ar, R¹ and R² are as defined in formula (Ia) and (Ib);

Compounds of general formula (Ia) and (Ib) can easily be prepared from the alcohols of general formula (IIIa) and (IIIb) respectively by either using the alcohol directly or pre-forming the alkoxide using a suitable base/reagent (e.g. NaH) and coupling to a suitably activated A-R¹ or R¹ group (or protected A-R¹ or R¹ group). Activated A-R¹ or R¹ group functionalities typically used for the formation of phosphates, esters, carbonates and carbamates include, but not limited to, phosphoryl chlorides, acid chlorides, activated carboxylic acids, chloroformates, activated carbonates and isocyanates. Alternatively, the A-R¹ or R¹ group can be introduced in a step-wise manner using standard methodologies. Suitable protecting group strategies can be employed where necessary. The formation of (Iaa) from (IIIa) using 2-dimethylaminoethyl carbonochloridate as an activated R group is representative of this approach.

The synthesis of Tonabersat, and other structurally related compounds, is disclosed in WO 95/34545. The present invention encompasses compounds prepared by applying the pro-drug groups -AR¹, R² and Q taught herein to the specific Examples disclosed in WO 95/34545. The methods proposed for the synthesis of compounds of general formula (I) are known to those skilled in the art, for example in Rautio et al., Nature Reviews Drug Discovery, 7, 255-270, 2008.

Optionally, a compound of formula (I) can also be transformed into another compound of formula (I) in one or more synthetic steps.

The following abbreviations have been used:
AcOH acetic acid
Ac₂O acetic anhydride
Ala L-Alanine
aq aqueous
Boc tertiary-butyloxycarbonyl
d day(s)
calcd calculated
DCC N,N′-Dicyclohexylcarbodiimide
DCM dichloromethane
DMAP 4-dimethylaminopyridine
DME dimethyl ether
DMF dimethylformamide
DMSO dimethyl sulfoxide
ES+, ESI+ electrospray ionization
EtOAc ethyl acetate
Et₂O diethyl ether
Et₃N triethylamine
Gly Glycine
h hour(s)
HATU (O-(7-azabenzotriazol-1-yl)-N,N,N′,N′-tetramethyluronium hexafluorophosphate)
HPLC High Performance Liquid Chromatography
HRMS High-Resolution Mass Spectrometry
Int Intermediate
LCMS Liquid Chromatography Mass Spectrometry
Leu L-Leucine
M molar
MeCN acetonitrile
MeOH methanol
MEK methyl ethyl ketone
MTBE methyl tertiary-butyl ether
[MH]⁺/[MH]⁻ protonated/deprotonated molecular ion
MS Mass Spectrometry
NIS N-iodosuccinimide
NMM N-methylmorpholine
Pro L-Proline
Rt retention time
sat saturated
THF tetrahydrofuran
Val L-Valine

### EXAMPLES AND INTERMEDIATE COMPOUNDS

Experimental Methods

Reactions were conducted at room temperature unless otherwise specified. Preparative chromatography was performed using a Flash Master Personal system equipped with Isolute Flash II silica columns or using a CombiFlash Companion system equipped with GraceResolv silica column, unless otherwise stated. The purest fractions were collected, concentrated and dried under vacuum. Compounds were typically dried in a vacuum oven at 40° C. prior to purity analysis. Compound analysis was performed by HPLC/LCMS using an Agilent 1100 HPLC system/Waters ZQ mass spectrometer connected to an Agilent 1100 HPLC system with a Phenomenex Synergi, RP-Hydro column (150×4.6 mm, 4 μm, 1.5 mL per min, 30° C., gradient 5-100% MeCN (+0.085% TFA) in water (+0.1% TFA) over 7 min, 200-300 nm). The compounds prepared were named using IUPAC nomenclature. Accurate masses were measured using a Waters QTOF electrospray ion source and corrected using Leucine Enkephalin lockmass. Spectra were acquired in positive and negative electrospray mode. The acquired mass range was m/z 100-1000. Samples were dissolved in DMSO to give 1 mg/mL solutions which were then further diluted with Acetonitrile (50%)/Water (50%) to 1 μg/mL solutions prior to analysis. The values reported correspond either to the protonated or deprotonated molecular ions [MH]⁺ or [MH]⁻.

### Intermediate 1

### N-[(3R,4S)-6-Acetyl-2,2-dimethyl-3-[(methylsulfanyl)methoxy]-3,4-dihydro-2H-1-benzopyran-4-yl]-4-fluorobenzamide

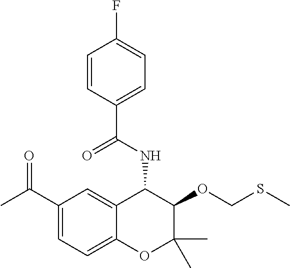 [SMILES: CSCO[C@@H]1[C@@H](NC(=O)c2ccc(F)cc2)c2cc(C(C)=O)ccc2OC1(C)C]

N-[(3R,4S)-6-Acetyl-3-hydroxy-2,2-dimethyl-3,4-dihydro-2H-1-benzopyran-4-yl]-4-fluorobenzamide (630 mg, 1.76 mmol) was dissolved in DMSO (1.17 mL), AcOH (2.07 mL, 36.2 mmol) and Ac₂O (898 uL, 9.52 mmol) were added and the reaction mixture was heated at 40-65° C. for 3.5 d. The reaction mixture was cooled to 0° C. and water (5 mL) was added drop-wise with stirring over 75 min. The precipitate was collected by filtration, washed with water (4 mL) and purified by column chromatography to give the title compound as a white solid (330 mg, 45%). HPLC: Rt 6.82 min, 98.8% purity.

### Intermediate 2

### N-[(3S,4S)-6-Acetyl-3-hydroxy-2,2-dimethyl-3,4-dihydro-2H-1-benzopyran-4-yl]-2-chlorobenzamide

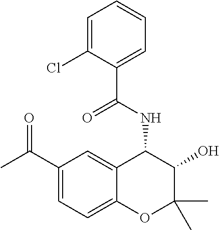 [SMILES: CC(=O)c1ccc2c(c1)[C@H](NC(=O)c1ccccc1Cl)[C@H](O)C(C)(C)O2]

2-Chlorobenzoyl chloride (2.19 g, 12.5 mmol) was added to a solution of 1-[(3S,4S)-4-amino-3-hydroxy-2,2-dimethyl-3,4-dihydro-2H-1-benzopyran-6-yl]ethan-1-one sulfuric acid hydrate (4.00 g, 11.4 mmol) and Et₃N (6.35 mL) in DCM (50 mL). The reaction mixture was stirred for 3 h, diluted with DCM (50 mL) and washed with 2M aq HCl (100 mL) and sat aq NaHCO₃ (100 mL), dried (MgSO₄) and concentrated in vacuo.

The residue was purified by column chromatography to give the title compound as an off-white solid (3.33 g, 78%). LCMS (ES⁺): 374.1 [MH]⁺.

### Intermediates 3-14

Intermediates 3-14 were prepared similarly to Intermediate 2, using the appropriate acid chloride and either 1-[(3S,4S)-4-amino-3-hydroxy-2,2-dimethyl-3,4-dihydro-2H-1-benzopyran-6-yl]ethan-1-one sulfuric acid hydrate or 1-[(3R,4S)-4-amino-3-hydroxy-2,2-dimethyl-3,4-dihydro-2H-1-benzopyran-6-yl]ethan-1-one hydrochloride; see Table 1 below.

TABLE 1
Amide formation

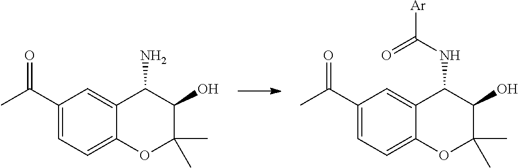

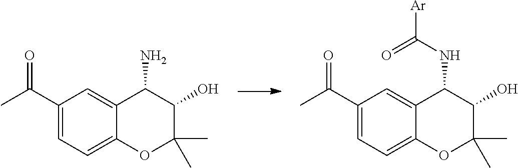

| | | | Form, |
|---|---|---|---|
| Int | Structure | Name | Yield, Analytical data |
| 3 | 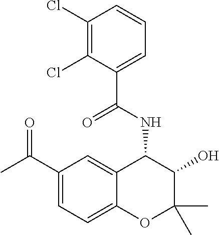 [SMILES: CC(=O)c1ccc2c(c1)[C@H](NC(=O)c1cccc(Cl)c1Cl)[C@H](O)C(C)(C)O2] | N-[(3S,4S)-6-Acetyl-3-hydroxy-2,2- dimethyl-3,4-dihydro-2H-1- benzopyran-4-yl]-2,3- dichlorobenzamide | Off-white solid. 4.04 g, 83% LCMS (ES⁺): 408.1 [MH]⁺ |
| 4 | 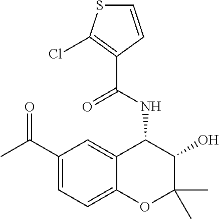 [SMILES: CC(=O)c1ccc2c(c1)[C@H](NC(=O)c1ccsc1Cl)[C@H](O)C(C)(C)O2] | N-[(3S,4S)-6-Acetyl-3-hydroxy-2,2- dimethyl-3,4-dihydro-2H-1- benzopyran-4-yl]-2-chlorothiophene- 3-carboxamide | Solid. 2.13 g, 49% LCMS (ES⁺): 380.0 [MH]⁺ |
| 5 | 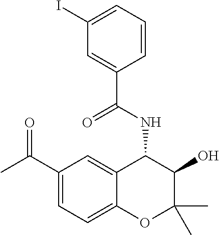 [SMILES: CC(=O)c1ccc2c(c1)[C@H](NC(=O)c1cccc(I)c1)[C@@H](O)C(C)(C)O2] | N-[(3R,4S)-6-Acetyl-3-hydroxy-2,2- dimethyl-3,4-dihydro-2H-1- benzopyran-4-yl]-3-iodobenzamide | White solid. 3.04 g, 89% LCMS (ES⁺): 466.0 [MH]⁺ |
| 6 | 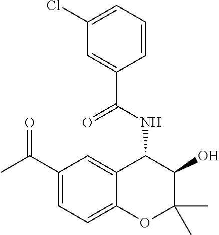 [SMILES: CC(=O)c1ccc2c(c1)[C@H](NC(=O)c1cccc(Cl)c1)[C@@H](O)C(C)(C)O2] | N-[(3R,4S)-6-Acetyl-3-hydroxy-2,2- dimethyl-3,4-dihydro-2H-1- benzopyran-4-yl]-3-chlorobenzamide | Solid 9.51 g, 86% LCMS (ES⁺): 374.1 [MH]⁺ |
| 7 | 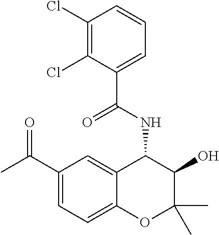 [SMILES: CC(=O)c1ccc2c(c1)[C@H](NC(=O)c1cccc(Cl)c1Cl)[C@@H](O)C(C)(C)O2] | N-[(3R,4S)-6-Acetyl-3-hydroxy-2,2- dimethyl-3,4-dihydro-2H-1- benzopyran-4-yl]-2,3- dichlorobenzamide | Solid. 1.95 g, 65% LCMS (ES⁺): 408.0 [MH]⁺ |
| 8 | 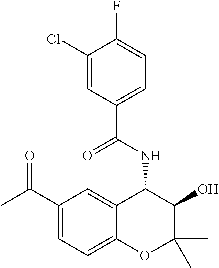 [SMILES: CC(=O)c1ccc2c(c1)[C@H](NC(=O)c1ccc(F)c(Cl)c1)[C@@H](O)C(C)(C)O2] | N-[(3R,4S)-6-Acetyl-3-hydroxy-2,2- dimethyl-3,4-dihydro-2H-1- benzopyran-4-yl]-3-chloro-4- fluorobenzamide | White solid. 2.67 g, 93% LCMS (ES⁺): 392.1 [MH]⁺ |
| 9 | 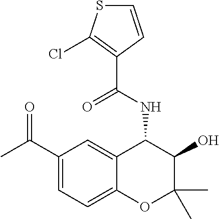 [SMILES: CC(=O)c1ccc2c(c1)[C@H](NC(=O)c1ccsc1Cl)[C@@H](O)C(C)(C)O2] | N-[(3R,4S)-6-Acetyl-3-hydroxy-2,2- dimethyl-3,4-dihydro-2H-1- benzopyran-4-yl]-2-chlorothiophene- 3-carboxamide | White solid. 2.80 g, 87% LCMS (ES⁺): 380.0 [MH]⁺ |
| 10 | 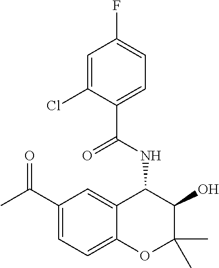 [SMILES: CC(=O)c1ccc2c(c1)[C@H](NC(=O)c1ccc(F)cc1Cl)[C@@H](O)C(C)(C)O2] | N-[(3R,4S)-6-Acetyl-3-hydroxy-2,2- dimethyl-3,4-dihydro-2H-1- benzopyran-4-yl]-2-chloro-4- fluorobenzamide | Off-white solid. 2.71 g, 94% LCMS (ES⁺): 392.1 [MH]⁺ |
| 11 | 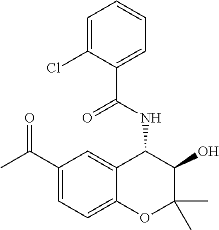 [SMILES: CC(=O)c1ccc2c(c1)[C@H](NC(=O)c1ccccc1Cl)[C@@H](O)C(C)(C)O2] | N-[(3R,4S)-6-Acetyl-3-hydroxy-2,2- dimethyl-3,4-dihydro-2H-1- benzopyran-4-yl]-2-chlorobenzamide | White solid. 2.70 g, 98% LCMS (ES⁺): 374.2 [MH]⁺ |
| 12 | 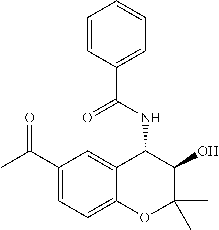 [SMILES: CC(=O)c1ccc2c(c1)[C@H](NC(=O)c1ccccc1)[C@@H](O)C(C)(C)O2] | N-[(3R,4S)-6-Acetyl-3-hydroxy-2,2- dimethyl-3,4-dihydro-2H-1- benzopyran-4-yl]benzamide | White solid. 2.30 g, 92% |
| 13 | 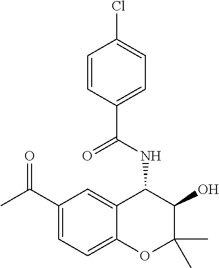 [SMILES: CC(=O)c1ccc2c(c1)[C@H](NC(=O)c1ccc(Cl)cc1)[C@@H](O)C(C)(C)O2] | N-[(3R,4S)-6-Acetyl-3-hydroxy-2,2- dimethyl-3,4-dihydro-2H-1- benzopyran-4-yl]-4-chlorobenzamide | Solid. 2.50 g, 91% LCMS (ES⁺): 374.2 [MH]⁺ |
| 14 | 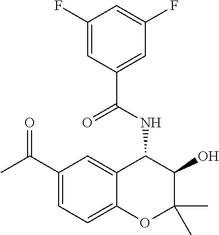 [SMILES: CC(=O)c1ccc2c(c1)[C@H](NC(=O)c1cc(F)cc(F)c1)[C@@H](O)C(C)(C)O2] | N-[(3R,4S)-6-Acetyl-3-hydroxy-2,2- dimethyl-3,4-dihydro-2H-1- benzopyran-4-yl]-3,5- difluorobenzamide | Solid. 2.60 g, 94% |

### Example 1

### {[(3R,4S)-6-Acetyl-4-[(4-fluorobenzene)amido]-2,2-dimethyl-3,4-dihydro-2H-1-benzopyran-3-yl]oxy}phosphonic acid

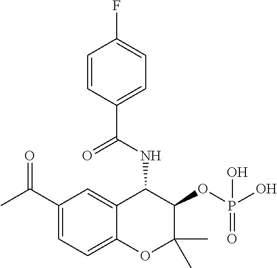 [SMILES: CC(=O)c1ccc2c(c1)[C@H](NC(=O)c1ccc(F)cc1)[C@@H](OP(=O)(O)O)C(C)(C)O2]

N-[(3R,4S)-6-Acetyl-3-hydroxy-2,2-dimethyl-3,4-dihydro-2H-1-benzopyran-4-yl]-4-fluorobenzamide (400 mg, 1.12 mmol) and pyridine (0.36 mL, 4.47 mmol) were dissolved in MEK (11 mL), POCl₃ (0.33 mL, 3.58 mmol) was added and the reaction mixture was stirred for 20 h. The precipitate was removed by filtration, washing with MEK (11 mL). 2M aq HCl (2 mL) was added and the reaction mixture was heated at 65° C. for 1 h and cooled to room temperature. The organic fraction was separated and washed with brine (5 mL), dried (MgSO₄) and concentrated in vacuo. The residue was slurried in EtOAc and dried to give the title compound (304 mg, 62%) as a white solid. HPLC: Rt 4.56 min, 94.3%. HRMS (ESI⁺) calcd for [MH]⁺ of C20H21FNO7P 436.0962, found 436.0962.

### Example 2

### Sodium {[(3R,4S)-6-Acetyl-4-[(4-fluorobenzene)amido]-2,2-dimethyl-3,4-dihydro-2H-1-benzopyran-3-yl]oxy}methyl hydrogen phosphate

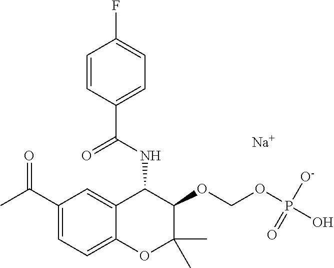 [SMILES: CC(=O)c1ccc2c(c1)[C@H](NC(=O)c1ccc(F)cc1)[C@@H](OCOP(=O)([O-])O)C(C)(C)O2 ; [Na+]]

Intermediate 1 (330 mg, 0.79 mmol) was dissolved in THF (3.5 mL), phosphoric acid (492 mg, 5.02 mmol) was added and the reaction mixture was cooled to 0° C. NIS (366 mg, 1.63 mmol) was added and the reaction mixture was stirred at 0° C. for 30 min. The reaction mixture was partitioned between EtOAc (3.5 mL) and 1M aq Na₂S₂O₃ (4 mL) and the organic fraction was washed with water (3 mL) and extracted into sat aq NaHCO₃ (×2), The combined aqueous fractions were washed with EtOAc (×4), diluted with EtOAc (3 mL) and cooled to 0° C. The reaction mixture was acidified to pH 1.48 with 2M aq HCl (800 uL) and the organic fraction was separated. 1M aq NaOH (0.25 mL, 0.25 mmol) was added and the reaction mixture was concentrated in vacuo to give the title compound (111 mg, 29%) as a white solid. HPLC: Rt 4.66 min, 97.9% purity. HRMS (ESI⁻) calcd for [M-H]⁻ of C21H22FNO8P 466.1067, found 466.1069.

### Example 3

### (3R,4S)-6-Acetyl-4-[(4-fluorobenzene)amido]-2,2-dimethyl-3,4-dihydro-2H-1-benzopyran-3-yl (2S)-2-amino-3-methylbutanoate hydrochloride

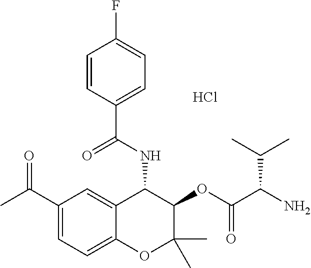 [SMILES: CC(=O)c1ccc2c(c1)[C@H](NC(=O)c1ccc(F)cc1)[C@@H](OC(=O)[C@@H](N)C(C)C)C(C)(C)O2 ; Cl]

N-[(3R,4S)-6-Acetyl-3-hydroxy-2,2-dimethyl-3,4-dihydro-2H-1-benzopyran-4-yl]-4-fluorobenzamide (893 mg, 2.50 mmol), N-Boc-Val (760 mg, 3.50 mmol) and DMAP (31.0 mg, 0.25 mmol) were dissolved in DCM (50 mL) and THF (10 mL), and a solution of DCC in DCM (3.75 mL, 1.0M, 3.75 mmol) was added at 0° C. The reaction mixture was stirred for 3 h and further DCC in DCM (1.00 mL, 1.0M, 1.00 mmol) was added. The reaction mixture was stirred for 1 h and concentrated in vacuo. The residue was dissolved in EtOAc, washed with 10% aq citric acid and brine, dried (MgSO₄) and concentrated in vacuo. The residue was purified by column chromatography, dissolved in DCM (8 mL) and MeOH (4 mL) and cooled to 0° C. 4M HCl in dioxane (15 mL) was added and the reaction mixture was stirred at 0° C. for 4 h and concentrated in vacuo. The residue was triturated from Et₂O to give the title compound (960 mg, 80%) as a white solid. HPLC: Rt 5.01 min, 98.0%. HRMS (ESI⁺) calcd for [MH]⁺ of C25H29FN2O5 457.2139, found 457.2150.

### Example 4

### (3R,4S)-6-Acetyl-4-[(4-fluorobenzene)amido]-2,2-dimethyl-3,4-dihydro-2H-1-benzopyran-3-yl (2S)-2-[(2S)-2-amino-3-methylbutanamido]-3-methylbutanoate hydrochloride

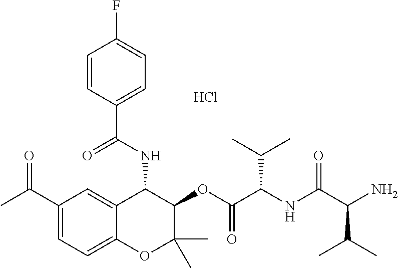 [SMILES: CC(=O)c1ccc2c(c1)[C@H](NC(=O)c1ccc(F)cc1)[C@@H](OC(=O)[C@@H](NC(=O)[C@@H](N)C(C)C)C(C)C)C(C)(C)O2 ; Cl]

N-Boc-Val (309 mg, 1.42 mmol) and HATU (648 mg, 1.70 mmol) were dissolved in DCM (15 mL) and DMF (1.5 mL) and the reaction mixture was stirred for 30 min. Example 3 (700 mg, 1.42 mmol) and NMM (0.47 mL, 4.26 mmol) were added and the reaction mixture was stirred for 5 h and concentrated in vacuo. The residue was partitioned between EtOAc and 10% aq citric acid and the resulting precipitate was collected by filtration and dried in vacuo. This material (700 mg) was dissolved in DCM (10 mL) and MeOH (5 mL) and a solution of 4M aq HCl in dioxane (10 mL) was added at 0° C. The reaction mixture was concentrated in vacuo and the residue was triturated from Et₂O and EtOAc and dried in vacuo to give the title compound (350 mg, 42%) as a white solid. HPLC: Rt 5.26 min, 99.1%. HRMS (ESI⁺) calcd for [MH]⁺ of C30H38FN3O6 556.2823, found 556.2798.

### Example 5

### (3R,4S)-6-Acetyl-4-[(4-fluorobenzene)amido]-2,2-dimethyl-3,4-dihydro-2H-1-benzopyran-3-yl 2-aminoacetate hydrochloride

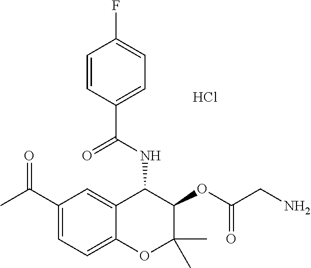 [SMILES: CC(=O)c1ccc2c(c1)[C@H](NC(=O)c1ccc(F)cc1)[C@@H](OC(=O)CN)C(C)(C)O2 ; Cl]

Example 5 was prepared similarly to Example 3, using N-Boc-Gly instead of N-Boc-Val, to give the title compound (143 mg, 32%) as a white solid. HPLC: Rt 4.65 min, 99.0%. HRMS (ESI⁺) calcd for [MH]⁺ of C22H23FN2O5 415.1669, found 415.1671.

### Example 6

### (3R,4S)-6-Acetyl-4-[(4-fluorobenzene)amido]-2,2-dimethyl-3,4-dihydro-2H-1-benzopyran-3-yl 2-[(2S)-pyrrolidin-2-ylformamido]acetate hydrochloride

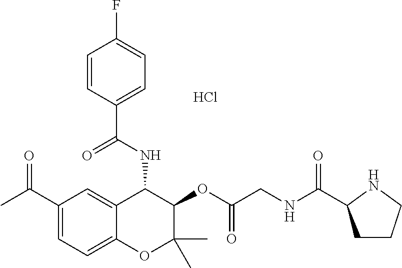 [SMILES: CC(=O)c1ccc2c(c1)[C@H](NC(=O)c1ccc(F)cc1)[C@@H](OC(=O)CNC(=O)[C@@H]1CCCN1)C(C)(C)O2 ; Cl]

Example 6 was prepared similarly to Example 3, using N-Boc-Pro-Gly instead of N-Boc-Val, to give the title compound (313 mg, 40%) as a white solid. HPLC: Rt 4.83 min, 99.5%. HRMS (ESI⁺) calcd for [MH]⁺ of C27H30FN3O6 512.2197, found 512.2194.

### Example 7

### (3R,4S)-6-Acetyl-4-[(4-fluorobenzene)amido]-2,2-dimethyl-3,4-dihydro-2H-1-benzopyran-3-yl 2-[(propan-2-yl)amino]acetate hydrochloride

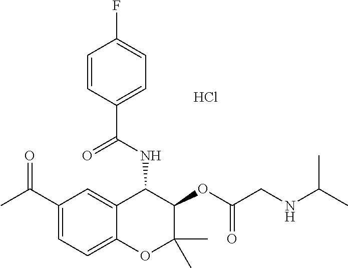 [SMILES: CC(=O)c1ccc2c(c1)[C@H](NC(=O)c1ccc(F)cc1)[C@@H](OC(=O)CNC(C)C)C(C)(C)O2 ; Cl]

Example 7 was prepared similarly to Example 3, using N-Boc-N-isopropylglycine instead of N-Boc-Val, to give the title compound (144 mg, 30%) as a white solid. HPLC: Rt 5.06 min, 100%. HRMS (ESI⁺) calcd for [MH]⁺ of C25H29FN2O5 457.2139, found 457.2136.

### Example 8

### (3R,4S)-6-Acetyl-4-[(4-fluorobenzene)amido]-2,2-dimethyl-3,4-dihydro-2H-1-benzopyran-3-yl 2-(morpholin-4-yl)ethyl carbonate hydrochloride

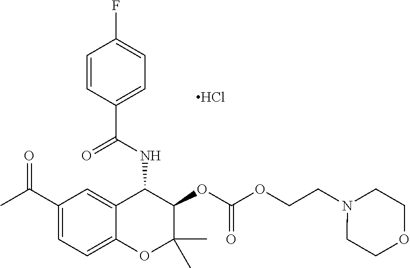 [SMILES: CC(=O)c1ccc2c(c1)[C@H](NC(=O)c1ccc(F)cc1)[C@@H](OC(=O)OCCN1CCOCC1)C(C)(C)O2 ; Cl]

N-[(3R,4S)-6-Acetyl-3-hydroxy-2,2-dimethyl-3,4-dihydro-2H-1-benzopyran-4-yl]-4-fluorobenzamide (357 mg, 1.00 mmol) and pyridine (175 uL, 2.20 mmol) were dissolved in DCM (13 mL) and triphosgene (99.0 mg, 0.33 mmol) was added. The reaction mixture was stirred for 1 h and N-(2-hydroxethyl)morpholine (130 uL,1.10 mmol) was added. The reaction mixture was stirred for 22 h, further N-(2-hydroxethyl)morpholine (1.00 mL, 8.46 mmol) was added and the reaction mixture was stirred for 25 h. Water (20 mL) was added and the aqueous fraction was separated and extracted with DCM (20 mL). The combined organic fractions were dried (MgSO₄) and concentrated in vacuo. The residue was purified by column chromatography, dissolved in DCM (4 mL), 2M HCl in Et₂O (1 mL) was added and the reaction mixture was concentrated in vacuo to give the title compound (141 mg, 26%) as a beige solid. HPLC: Rt 4.98 min, 99.3%. HRMS (ESI⁺) calcd for [MH]⁺of C27H31FN2O7 515.2194, found 515.2195.

### Examples 9-21

Examples 9-21 were prepared similarly to Example 1 using Intermediates 2-14; see Table 2 below.

TABLE 2
Phosphate ester formation

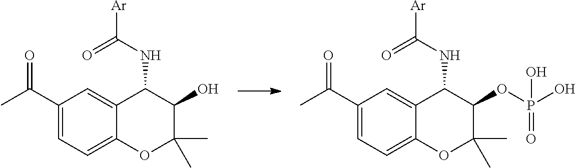

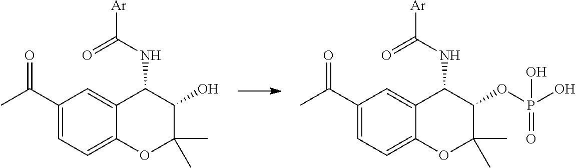

| | | | Intermediate(s), Form, |
|---|---|---|---|
| Ex | Structure | Name | Yield, Analytical data |
| 9 | 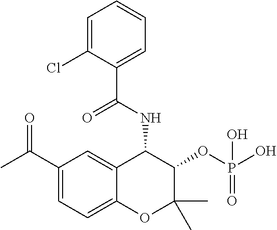 [SMILES: CC(=O)c1ccc2c(c1)[C@H](NC(=O)c1ccccc1Cl)[C@H](OP(=O)(O)O)C(C)(C)O2] | {[(3S,4S)-6-Acetyl-4-[(2- chlorobenzene)amido]-2,2-dimethyl- 3,4-dihydro-2H-1-benzopyran-3- yl]oxy}phosphonic acid | Using Intermediate 2 Off-white solid. 206 mg, 42% HPLC: Rt 4.54 min, 99.6%. HRMS (ESI⁺) calcd for [MH]⁺ of C20H21ClNO7P 452.0666, found 452.0668 |
| 10 | 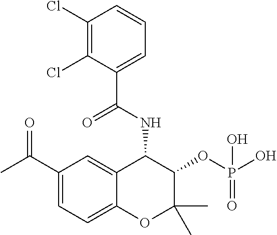 [SMILES: CC(=O)c1ccc2c(c1)[C@H](NC(=O)c1cccc(Cl)c1Cl)[C@H](OP(=O)(O)O)C(C)(C)O2] | {[(3S,4S)-6-Acetyl-4-[(2,3- dichlorobenzene)amido]-2,2-dimethyl- 3,4-dihydro-2H-1-benzopyran-3- yl]oxy}phosphonic acid | Using Intermediate 3 Off-white solid. 242 mg, 50% HPLC: Rt 4.95 min, 100%. HRMS (ESI⁺) calcd for [MH]⁺ of C20H20Cl2NO7P 486.0276, found 486.0275 |
| 11 | 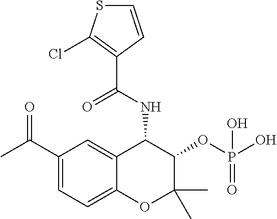 [SMILES: CC(=O)c1ccc2c(c1)[C@H](NC(=O)c1ccsc1Cl)[C@H](OP(=O)(O)O)C(C)(C)O2] | {[(3S,4S)-6-Acetyl-4-(2- chlorothiophene-3-amido)-2,2- dimethyl-3,4-dihydro-2H-1- benzopyran-3-yl]oxy}phosphonic acid | Using Intermediate 4 Off-white solid. 152 mg, 65% HPLC: Rt 4.54 min, 100%. HRMS (ESI⁺) calcd for [MH]⁺ of C18H19ClNO7PS 458.0230, found 458.0227 |
| 12 | 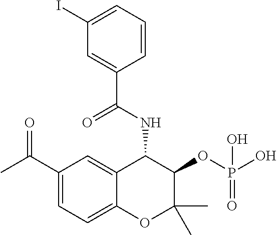 [SMILES: CC(=O)c1ccc2c(c1)[C@H](NC(=O)c1cccc(I)c1)[C@@H](OP(=O)(O)O)C(C)(C)O2] | {[(3R,4S)-6-Acetyl-4-[(3- iodobenzene)amido]-2,2-dimethyl- 3,4-dihydro-2H-1-benzopyran-3- yl]oxy}phosphonic acid | Using Intermediate 5 Off-white solid. 322 mg, 69% HPLC: Rt 5.02 min, 97.2%. HRMS (ESI⁺) calcd for [MH]⁺ of C20H21lNO7P 544.0022, found 544.0020 |
| 13 | 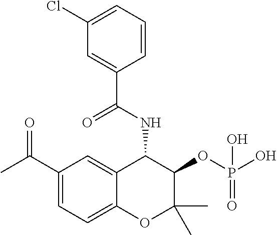 [SMILES: CC(=O)c1ccc2c(c1)[C@H](NC(=O)c1cccc(Cl)c1)[C@@H](OP(=O)(O)O)C(C)(C)O2] | {[(3R,4S)-6-Acetyl-4-[(3- chlorobenzene)amido]-2,2-dimethyl- 3,4-dihydro-2H-1-benzopyran-3- yl]oxy}phosphonic acid | Using Intermediate 6 White solid. 383 mg, 79% HPLC: Rt 4.84 min, 99.0%. HRMS (ESI⁺) calcd for [MH]⁺ of C20H21ClNO7P 452.0666, found 452.0660 |
| 14 | 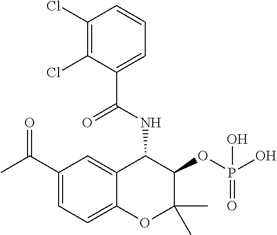 [SMILES: CC(=O)c1ccc2c(c1)[C@H](NC(=O)c1cccc(Cl)c1Cl)[C@@H](OP(=O)(O)O)C(C)(C)O2] | {[(3R,4S)-6-Acetyl-4-[(2,3- dichlorobenzene)amido]-2,2-dimethyl- 3,4-dihydro-2H-1-benzopyran-3- yl]oxy}phosphonic acid | Using Intermediate 7 White solid. 108 mg, 23% HPLC: Rt 4.96 min, 98.4%. HRMS (ESI⁺) calcd for [MH]⁺ of C20H20Cl2NO7P 486.0276, found 486.0273 |
| 15 | 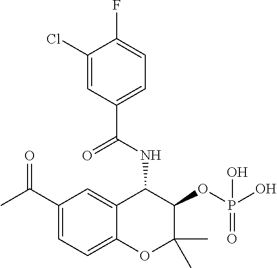 [SMILES: CC(=O)c1ccc2c(c1)[C@H](NC(=O)c1ccc(F)c(Cl)c1)[C@@H](OP(=O)(O)O)C(C)(C)O2] | {[(3R,4S)-6-Acetyl-4-[(3-chloro-4- fluorobenzene)amido]-2,2-dimethyl- 3,4-dihydro-2H-1-benzopyran-3- yl]oxy}phosphonic acid | Using Intermediate 8 White solid. 204 mg, 42% HPLC: Rt 4.92 min, 100%. HRMS (ESI⁺) calcd for [MH]⁺ of C20H20ClFNO7P 470.0572, found 470.0580 |
| 16 | 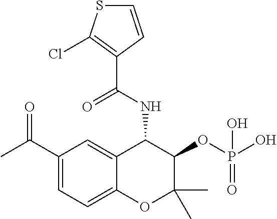 [SMILES: CC(=O)c1ccc2c(c1)[C@H](NC(=O)c1ccsc1Cl)[C@@H](OP(=O)(O)O)C(C)(C)O2] | {[(3R,4S)-6-Acetyl-4-(2- chlorothiophene-3-amido)-2,2- dimethyl-3,4-dihydro-2H-1- benzopyran-3-yl]oxy}phosphonic acid | Using Intermediate 9 White solid. 182 mg, 38% HPLC: Rt 4.61 min, 95.6%. HRMS (ESI⁺) calcd for [MH]⁺ of C18H19ClNO7PS 458.0230, found 458.0219 |
| 17 | 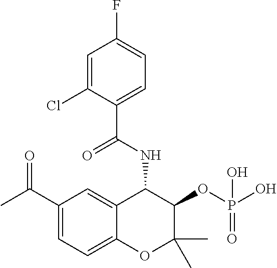 [SMILES: CC(=O)c1ccc2c(c1)[C@H](NC(=O)c1ccc(F)cc1Cl)[C@@H](OP(=O)(O)O)C(C)(C)O2] | {[(3R,4S)-6-Acetyl-4-[(2-chloro-4- fluorobenzene)amido]-2,2-dimethyl- 3,4-dihydro-2H-1-benzopyran-3- yl]oxy}phosphonic acid | Using Intermediate 10 White solid. 311 mg, 65% HPLC: Rt 4.72 min, 98.7%. HRMS (ESI⁺) calcd for [MH]⁺ of C20H20ClFNO7P 470.0572, found 470.0568 |
| 18 | 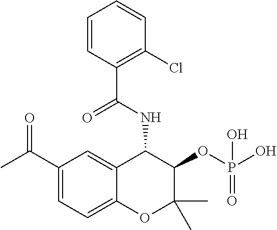 [SMILES: CC(=O)c1ccc2c(c1)[C@H](NC(=O)c1ccccc1Cl)[C@@H](OP(=O)(O)O)C(C)(C)O2] | {[(3R,4S)-6-Acetyl-4-[(2- chlorobenzene)amido]-2,2-dimethyl- 3,4-dihydro-2H-1-benzopyran-3- yl]oxy}phosphonic acid | Using Intermediate 11 Off white solid. 77.0 mg, 13% HPLC: Rt 4.58 min, 99.7%. HRMS (ESI⁺) calcd for [MH]⁺ of C20H21ClNO7P 452.0666, found 452.0662 |
| 19 | 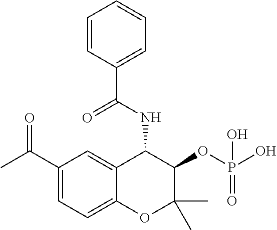 [SMILES: CC(=O)c1ccc2c(c1)[C@H](NC(=O)c1ccccc1)[C@@H](OP(=O)(O)O)C(C)(C)O2] | {[(3R,4S)-6-Acetyl-4-benzamido-2,2- dimethyl-3,4-dihydro-2H-1- benzopyran-3-yl]oxy}phosphonic acid | Using Intermediate 12 White solid. 160 mg, 32% HPLC: Rt 4.44 min, 98.2%. HRMS (ESI⁺) calcd for [MH]⁺ of C20H22NO7P 418.1056, found 418.1049 |
| 20 | 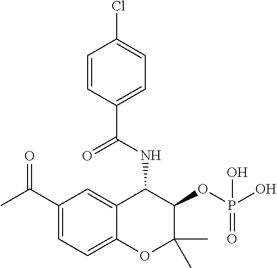 [SMILES: CC(=O)c1ccc2c(c1)[C@H](NC(=O)c1ccc(Cl)cc1)[C@@H](OP(=O)(O)O)C(C)(C)O2] | {[(3R,4S)-6-Acetyl-4-[(4- chlorobenzene)amido]-2,2-dimethyl- 3,4-dihydro-2H-1-benzopyran-3- yl]oxy}phosphonic acid | Using Intermediate 13 White solid. 486 mg, 77% HPLC: Rt 4.84 min, 100%. HRMS (ESI⁺) calcd for [MH]⁺ of C20H21ClNO7P 452.0666, found 452.0668 |
| 21 | 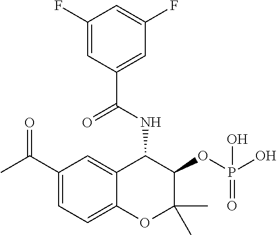 [SMILES: CC(=O)c1ccc2c(c1)[C@H](NC(=O)c1cc(F)cc(F)c1)[C@@H](OP(=O)(O)O)C(C)(C)O2] | {[(3R,4S)-6-Acetyl-4-[(3,5- difluorobenzene)amido]-2,2-dimethyl- 3,4-dihydro-2H-1-benzopyran-3- yl]oxy}phosphonic acid | Using Intermediate 14 White solid. 180 mg, 37% HPLC: Rt 4.74 min, 97.2%. HRMS (ESI⁺) calcd for [MH]⁺ of C20H20F2NO7P 454.0867, found 454.0867 |

### Examples 22-34

Examples 22-34 were prepared similarly to Example 2 using Intermediates 2-14; see Table 3 below.

TABLE 3
Phosphoryloxymethyl prodrug formation

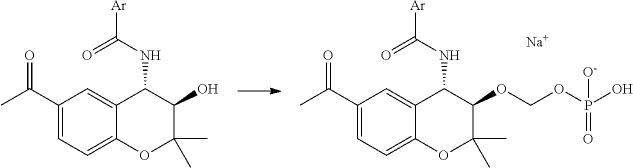

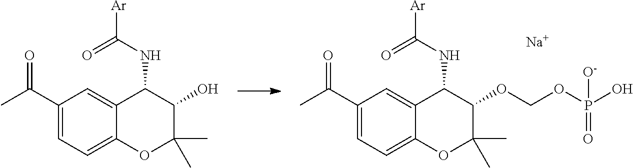

| | | | Intermediate(s), Form, |
|---|---|---|---|
| Ex | Structure | Name | Yield, Analytical data |
| 22 | 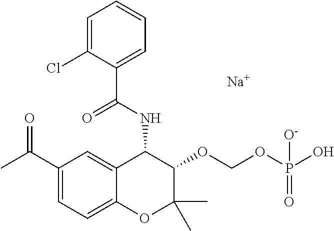 [SMILES: CC(=O)c1ccc2c(c1)[C@H](NC(=O)c1ccccc1Cl)[C@H](OCOP(=O)([O-])O)C(C)(C)O2 ; [Na+]] | Sodium {[(3S,4S)-6-acetyl-4-[(2- chlorobenzene)amido]-2,2-dimethyl- 3,4-dihydro-2H-1-benzopyran-3- yl]oxy}methyl hydrogen phosphate | Using Intermediate 2 Off-white solid. 1.00 g, 25% HPLC: Rt 4.60 min, 96.7%. HRMS (ESI⁻) calcd for [M − H]⁻ of C21H22ClNO8P 482.0772, found 482.0774 |
| 23 | 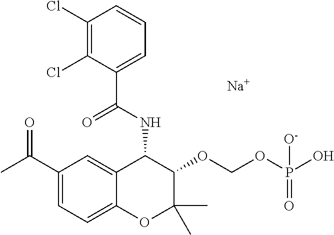 [SMILES: CC(=O)c1ccc2c(c1)[C@H](NC(=O)c1cccc(Cl)c1Cl)[C@H](OCOP(=O)([O-])O)C(C)(C)O2 ; [Na+]] | Sodium {[(3S,4S)-6-acetyl-4-[(2,3- dichlorobenzene)amido]-2,2-dimethyl- 3,4-dihydro-2H-1-benzopyran-3- yl]oxy}methyl hydrogen phosphate | Using Intermediate 3 White solid. 401 mg, 15% HPLC: Rt 4.97 min, 98.9%.]⁻ HRMS (ESI⁻) calcd for [M − H]⁻ of C21H21Cl2NO8P 516.0382, found 516.0378 |
| 24 | 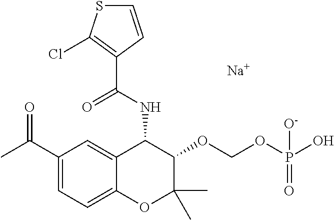 [SMILES: CC(=O)c1ccc2c(c1)[C@H](NC(=O)c1ccsc1Cl)[C@H](OCOP(=O)([O-])O)C(C)(C)O2 ; [Na+]] | Sodium {[(3S,4S)-6-acetyl-4-(2- chlorothiophene-3-amido)-2,2- dimethyl-3,4-dihydro-2H-1- benzopyran-3-yl]oxy}methyl hydrogen phosphate | Using Intermediate 4 White solid. 59.0 mg, 5% HPLC: Rt 4.67 min, 100%. HRMS (ESI⁻) calcd for [M − H]⁻ of C19H20ClNO8PS 488.0336, found 488.0335 |
| 25 | 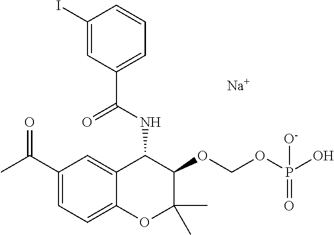 [SMILES: CC(=O)c1ccc2c(c1)[C@H](NC(=O)c1cccc(I)c1)[C@@H](OCOP(=O)([O-])O)C(C)(C)O2 ; [Na+]] | Sodium {[(3R,4S)-6-acetyl-4-[(3- iodobenzene)amido]-2,2-dimethyl-3,4- dihydro-2H-1-benzopyran-3- yl]oxy}methyl hydrogen phosphate | Using Intermediate 5 White solid. 414 mg, 20% HPLC: Rt 5.10 min, 98.0%. HRMS (ESI⁻) calcd for [M − H]⁻ of C21H22lNO8P 574.0128, found 574.0131 |
| 26 | 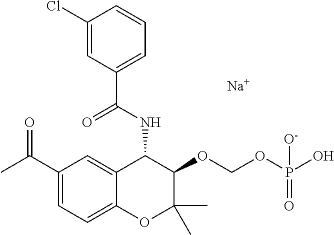 [SMILES: CC(=O)c1ccc2c(c1)[C@H](NC(=O)c1cccc(Cl)c1)[C@@H](OCOP(=O)([O-])O)C(C)(C)O2 ; [Na+]] | Sodium {[(3R,4S)-6-acetyl-4-[(3- chlorobenzene)amido]-2,2-dimethyl- 3,4-dihydro-2H-1-benzopyran-3- yl]oxy}methyl hydrogen phosphate | Using Intermediate 6 Cream solid. 78.0 mg, 6% HPLC: Rt 4.91 min, 98.9%. HRMS (ESI⁻) calcd for [M − H]⁻ of C21H22ClNO8P 482.0772, found 482.0772 |
| 27 | 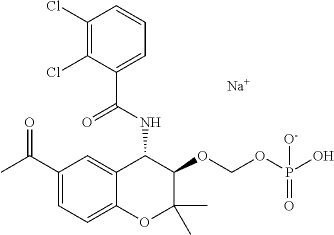 [SMILES: CC(=O)c1ccc2c(c1)[C@H](NC(=O)c1cccc(Cl)c1Cl)[C@@H](OCOP(=O)([O-])O)C(C)(C)O2 ; [Na+]] | Sodium {[(3R,4S)-6-acetyl-4-[(2,3- dichlorobenzene)amido]-2,2-dimethyl- 3,4-dihydro-2H-1-benzopyran-3- yl]oxy}methyl hydrogen phosphate | Using Intermediate 7 White solid. 110 mg, 8.3% HPLC: Rt 4.97 min, 99.3%. HRMS (ESI⁻) calcd for [M − H]⁻ of C21H21Cl2NO8P 516.0382, found 516.0385 |
| 28 | 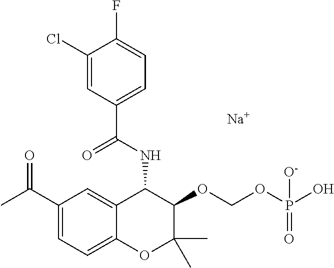 [SMILES: CC(=O)c1ccc2c(c1)[C@H](NC(=O)c1ccc(F)c(Cl)c1)[C@@H](OCOP(=O)([O-])O)C(C)(C)O2 ; [Na+]] | Sodium {[(3R,4S)-6-acetyl-4-[(3- chloro-4-fluorobenzene)amido]-2,2- dimethyl-3,4-dihydro-2H-1- benzopyran-3-yl]oxy}methyl hydrogen phosphate | Using Intermediate 8 White solid. 460 mg, 23% HPLC: Rt 5.02 min, 98.1%. HRMS (ESI⁻) calcd for [M − H]⁻ of C21H21ClFNO8P 500.0677, found 500.0675 |
| 29 | 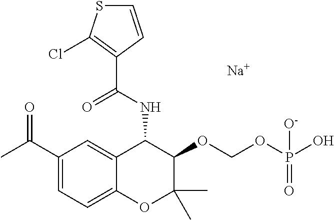 [SMILES: CC(=O)c1ccc2c(c1)[C@H](NC(=O)c1ccsc1Cl)[C@@H](OCOP(=O)([O-])O)C(C)(C)O2 ; [Na+]] | Sodium {[(3R,4S)-6-acetyl-4-(2- chlorothiophene-3-amido)-2,2- dimethyl-3,4-dihydro-2H-1- benzopyran-3-yl]oxy}methyl hydrogen phosphate | Using Intermediate 9 White solid. 491 mg, 24% HPLC: Rt 4.67 min, 99.4%. HRMS (ESI⁻) calcd for [M − H]⁻ of C19H20ClNO8PS 488.0336, found 488.0334 |
| 30 | 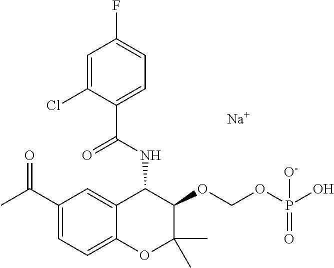 [SMILES: CC(=O)c1ccc2c(c1)[C@H](NC(=O)c1ccc(F)cc1Cl)[C@@H](OCOP(=O)([O-])O)C(C)(C)O2 ; [Na+]] | Sodium {[(3R,4S)-6-acetyl-4-[(2- chloro-4-fluorobenzene)amido]-2,2- dimethyl-3,4-dihydro-2H-1- benzopyran-3-yl]oxy}methyl hydrogen phosphate | Using Intermediate 10 White solid. 444 mg, 22% HPLC: Rt 4.74 min, 98.1%. HRMS (ESI⁻) calcd for [M − H]⁻ of C21H21ClFNO8P 500.0677, found 500.0685 |
| 31 | 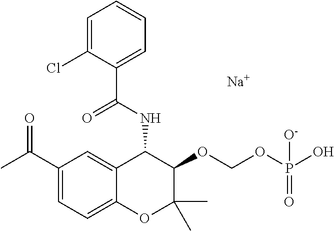 [SMILES: CC(=O)c1ccc2c(c1)[C@H](NC(=O)c1ccccc1Cl)[C@@H](OCOP(=O)([O-])O)C(C)(C)O2 ; [Na+]] | Sodium {[(3R,4S)-6-acetyl-4-[(2- chlorobenzene)amido]-2,2-dimethyl- 3,4-dihydro-2H-1-benzopyran-3- yl]oxy}methyl hydrogen phosphate | Using Intermediate 11 Off white solid. 113 mg, 7.1% HPLC: Rt 4.62 min, 97.1%. HRMS (ESI⁻) calcd for [M − H]⁻ of C21H22ClNO8P 482.0772, found 482.0770 |
| 32 | 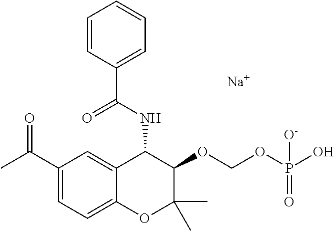 [SMILES: CC(=O)c1ccc2c(c1)[C@H](NC(=O)c1ccccc1)[C@@H](OCOP(=O)([O-])O)C(C)(C)O2 ; [Na+]] | Sodium {[(3R,4S)-6-acetyl-4- benzamido-2,2-dimethyl-3,4-dihydro- 2H-1-benzopyran-3-yl]oxy}methyl hydrogen phosphate | Using Intermediate 12 White solid. 112 mg, 13% HPLC: Rt 4.48 min, 100%. HRMS (ESI⁻) calcd for [M − H]⁻ of C21H23NO8P 448.1161, found 448.1164 |
| 33 | 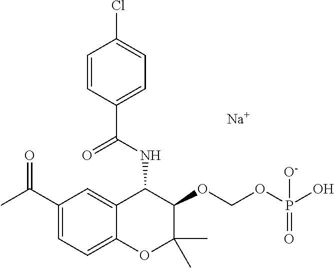 [SMILES: CC(=O)c1ccc2c(c1)[C@H](NC(=O)c1ccc(Cl)cc1)[C@@H](OCOP(=O)([O-])O)C(C)(C)O2 ; [Na+]] | Sodium {[(3R,4S)-6-acetyl-4-[(4- chlorobenzene)amido]-2,2-dimethyl- 3,4-dihydro-2H-1-benzopyran-3- yl]oxy}methyl hydrogen phosphate | Using Intermediate 13 White solid. 230 mg, 8% HPLC: Rt 4.89 min, 97.0%. HRMS (ESI⁻) calcd for [M − H]⁻ of C21H22ClNO8P 482.0772, found 482.0778 |
| 34 | 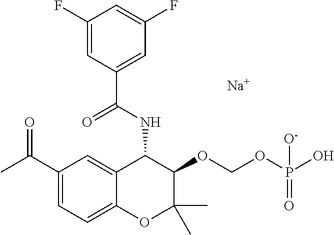 [SMILES: CC(=O)c1ccc2c(c1)[C@H](NC(=O)c1cc(F)cc(F)c1)[C@@H](OCOP(=O)([O-])O)C(C)(C)O2 ; [Na+]] | Sodium {[(3R,4S)-6-acetyl-4-[(3,5- difluorobenzene)amido]-2,2-dimethyl- 3,4-dihydro-2H-1-benzopyran-3- yl]oxy}methyl hydrogen phosphate | Using Intermediate 14 White solid. 173 mg, 14% HPLC: Rt 4.81 min, 98.6%. HRMS (ESI⁻) calcd for [M − H]⁻ of C21H21F2NO8P 484.0973, found 484.0976 |

### Example 35

### (3R,4S)-6-Acetyl-4-[(3-chlorobenzene)amido]-2,2-dimethyl-3,4-dihydro-2H-1-benzopyran-3-yl (2S)-2-amino-3-methylbutanoate hydrochloride

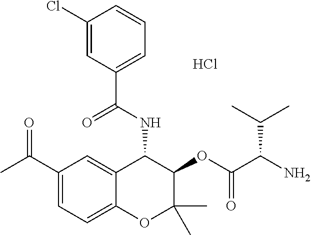 [SMILES: CC(=O)c1ccc2c(c1)[C@H](NC(=O)c1cccc(Cl)c1)[C@@H](OC(=O)[C@@H](N)C(C)C)C(C)(C)O2 ; Cl]

Example 35 was prepared similarly to Example 3, using Intermediate 6 instead of N-[(3R,4S)-6-acetyl-3-hydroxy-2,2-dimethyl-3,4-dihydro-2H-1-benzopyran-4-yl]-4-fluorobenzamide, to give the title compound (800 mg, 59%) as a white solid. HPLC: Rt 5.26 min, 100%. HRMS (ESI⁺) calcd for [MH]⁺ of C25H29ClN2O5 473.1843, found 473.1833.

### Example 36

### (3R,4S)-6-Acetyl-4-[(3-chlorobenzene)amido]-2,2-dimethyl-3,4-dihydro-2H-1-benzopyran-3-yl (2S)-2-[(2S)-2-amino-3-methylbutanamido]-3-methylbutanoate hydrochloride

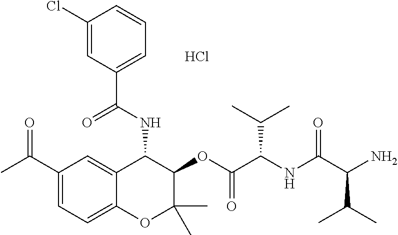 [SMILES: CC(=O)c1ccc2c(c1)[C@H](NC(=O)c1cccc(Cl)c1)[C@@H](OC(=O)[C@@H](NC(=O)[C@@H](N)C(C)C)C(C)C)C(C)(C)O2 ; Cl]

Example 36 was prepared similarly to Example 4, using Example 35 instead of Example 3, to give the title compound (390 mg, 47%) as a white solid. HPLC: Rt 5.53 min, 98.2%. HRMS (ESI⁺) calcd for [MH]⁺ of C30H38ClN3O6 572.2527, found 572.2531.

### Example 37

### (3R,4S)-6-Acetyl-4-[(3-chlorobenzene)amido]-2,2-dimethyl-3,4-dihydro-2H-1-benzopyran-3-yl (2S)-2-amino-4-methylpentanoate hydrochloride

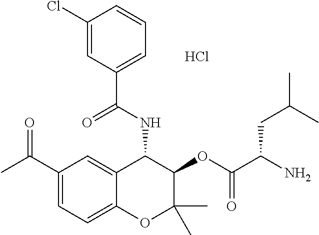 [SMILES: CC(=O)c1ccc2c(c1)[C@H](NC(=O)c1cccc(Cl)c1)[C@@H](OC(=O)[C@@H](N)CC(C)C)C(C)(C)O2 ; Cl]

Example 37 was prepared similarly to Example 35, using N-Boc-Leu instead of N-Boc-Val, to give the title compound (301 mg, 29%) as a white solid. HPLC: Rt 5.50 min, 99.8%. HRMS (ESI⁺) calcd for [MH]⁺ of C26H31ClN2O5 487.2000, found 487.2000.

### Example 38

### (3R,4S)-6-Acetyl-4-[(3-chlorobenzene)amido]-2,2-dimethyl-3,4-dihydro-2H-1-benzopyran-3-yl (2R)-2-amino-4-methylpentanoate hydrochloride

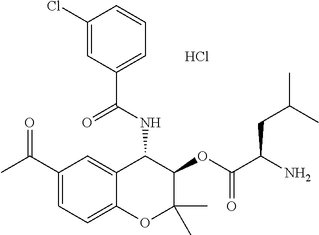 [SMILES: CC(=O)c1ccc2c(c1)[C@H](NC(=O)c1cccc(Cl)c1)[C@@H](OC(=O)[C@H](N)CC(C)C)C(C)(C)O2 ; Cl]

Example 38 was prepared similarly to Example 37, using N-Boc-D-Leucine instead of N-Boc-L-Leucine, to give the title compound (350 mg, 56%) as a white solid. HPLC: Rt 5.59 min, 98.0%. HRMS (ESI⁺) calcd for [MH]⁺ of C26H31ClN2O5 487.2000, found 487.2003.

### Example 39

### (3R,4S)-6-Acetyl-4-[(3-chlorobenzene)amido]-2,2-dimethyl-3,4-dihydro-2H-1-benzopyran-3-yl 2-[(propan-2-yl)amino]acetate hydrochloride

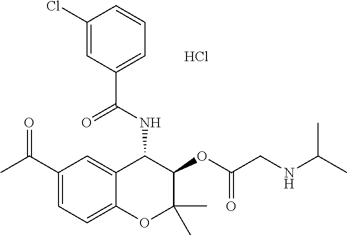 [SMILES: CC(=O)c1ccc2c(c1)[C@H](NC(=O)c1cccc(Cl)c1)[C@@H](OC(=O)CNC(C)C)C(C)(C)O2 ; Cl]

Example 39 was prepared similarly to Example 35, using N-Boc-N-isopropylglycine instead of N-Boc-Val, to give the title compound (411 mg, 52%) as a white solid. HPLC: Rt 5.31 min, 100%. HRMS (ESI⁺) calcd for [MH]⁺ of C25H29ClN2O5 473.1843, found 473.1837.

### Example 40

### (3R,4S)-6-Acetyl-4-[(3-chlorobenzene)amido]-2,2-dimethyl-3,4-dihydro-2H-1-benzopyran-3-yl (2S)-2-aminopropanoate hydrochloride

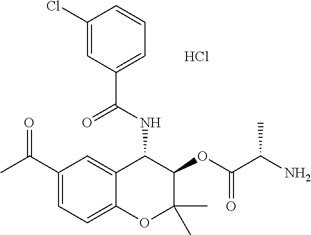 [SMILES: CC(=O)c1ccc2c(c1)[C@H](NC(=O)c1cccc(Cl)c1)[C@@H](OC(=O)[C@H](C)N)C(C)(C)O2 ; Cl]

Example 40 was prepared similarly to Example 35, using N-Boc-Ala instead of N-Boc-Val, to give the title compound (3.38 g, 82%) as a white solid. HPLC: Rt 4.99 min, 98.9%. HRMS (ESI⁺) calcd for [MH]⁺ of C23H25ClN2O5 445.1530, found 445.1533.

### Example 41

### (3R,4S)-6-Acetyl-4-[(3-chlorobenzene)amido]-2,2-dimethyl-3,4-dihydro-2H-1-benzopyran-3-yl (2S)-2-(methylamino)propanoate hydrochloride

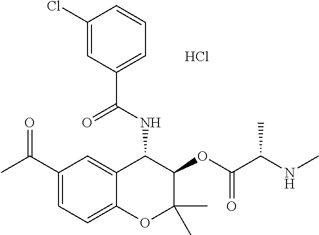 [SMILES: CN[C@@H](C)C(=O)O[C@@H]1[C@@H](NC(=O)c2cccc(Cl)c2)c2cc(C(C)=O)ccc2OC1(C)C ; Cl]

Example 41 was prepared similarly to Example 35, using N-Boc-N-Me-Ala instead of N-Boc-Val, to give the title compound (1.01 g, 76%) as a white solid. HPLC: Rt 5.10 min, 100%. HRMS (ESI⁺) calcd for [MH]⁺ of C24H27ClN2O5 459.1687, found 459.1680.

### Example 42

### (3R,4S)-6-Acetyl-4-[(3-chlorobenzene)amido]-2,2-dimethyl-3,4-dihydro-2H-1-benzopyran-3-yl (2S)-2-[(2S)-2-amino-3-methylbutanamido]propanoate hydrochloride

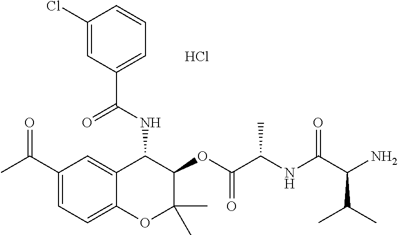 [SMILES: CC(=O)c1ccc2c(c1)[C@H](NC(=O)c1cccc(Cl)c1)[C@@H](OC(=O)[C@H](C)NC(=O)[C@@H](N)C(C)C)C(C)(C)O2 ; Cl]

Example 42 was prepared similarly to Example 36, using Example 40 instead of Example 35, to give the title compound (731 mg, 63%) as a white solid. HPLC: Rt 5.23 min, 97.8%. HRMS (ESI⁺) calcd for [MH]⁺ of C28H34ClN3O6 544.2214, found 544.2216.

### Example 43

### (3R,4S)-6-Acetyl-4-[(3-chlorobenzene)amido]-2,2-dimethyl-3,4-dihydro-2H-1-benzopyran-3-yl (2S)-2-[(2S)-2-amino-N,3-dimethylbutanamido]propanoate hydrochloride

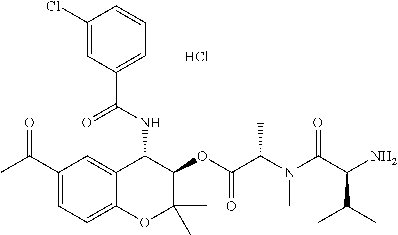 [SMILES: CC(=O)c1ccc2c(c1)[C@H](NC(=O)c1cccc(Cl)c1)[C@@H](OC(=O)[C@H](C)N(C)C(=O)[C@@H](N)C(C)C)C(C)(C)O2 ; Cl]

Example 43 was prepared similarly to Example 36, using Example 41 instead of Example 35, to give the title compound (331 mg, 30%) as a white solid. HPLC: Rt 5.40 min, 100%. HRMS (ESI⁺) calcd for [MH]⁺ of C29H36ClN3O6 558.2371, found 558.2375.

### Example 44

### (3R,4S)-6-Acetyl-4-[(3-chlorobenzene)amido]-2,2-dimethyl-3,4-dihydro-2H-1-benzopyran-3-yl (2S)-2-[(2S)-2-amino-4-methylpentanamido]propanoate hydrochloride

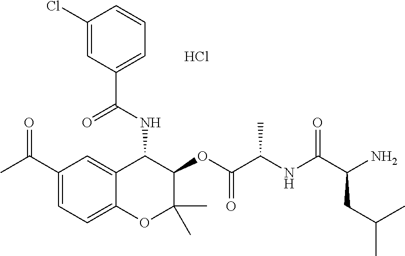 [SMILES: CC(=O)c1ccc2c(c1)[C@H](NC(=O)c1cccc(Cl)c1)[C@@H](OC(=O)[C@H](C)NC(=O)[C@@H](N)CC(C)C)C(C)(C)O2 ; Cl]

Example 44 was prepared similarly to Example 42, using N-Boc-Leu instead of N-Boc-Val, to give the title compound (798 mg, 67%) as a white solid. HPLC: Rt 5.44 min, 97.8%. HRMS (ESI⁺) calcd for [MH]⁺ of C29H36ClN3O6 558.2371, found 558.2378.

Pharmacokinetics

Example Prodrugs of the claimed invention were dosed either intravenously or orally to fasted male Sprague Dawley rats. The rats underwent surgery for jugular vein cannulation 48 h prior to dosing. Following dosing, 0.25 mL blood samples were taken via the cannulae at 0, 5, 10, 20, 30, 45, 60, 120, 240 & 360 min in EDTA coated tubes. Tubes were spun at 13,000 rpm for 4 min and 100 ul of supernatant taken immediately and stored at −80° C. prior to analysis. Plasma samples were analysed by LC-MS/MS following extraction by protein precipitation, and levels of parent prodrug and the hydrolysis product drug (e.g. tonabersat) were measured by MRM (Multiple Reaction Monitoring) analysis against an extracted calibration curve of plasma samples spiked with the Example prodrug and the corresponding drug.

Scheme 6 shows the in vivo hydrolysis of prodrug compounds of the invention of formula (Ia) to the corresponding drug of formula (IIIa). Similarly, prodrugs of formula (Ib) hydrolyse to drugs of formula (IIIb).

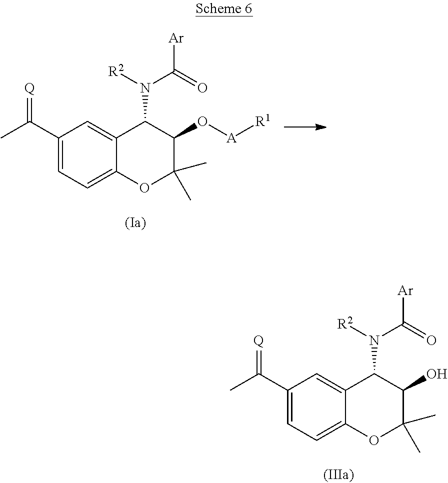

The exposure of the drug of formula (IIIa) or (IIIb) (including, for example, tonabersat or carabersat) in plasma following dosing of the prodrugs of the invention was compared directly to the exposure observed following dosing of an equimolar amount of the same drug of formula (IIIa) or (IIIb) under analogous assay conditions (5.00 mg/kg oral dosing or 0.78 mg/kg intravenous dosing). In an embodiment prodrugs of the present invention have >10% exposure of the drug of formula (IIIa) or (IIIb) obtained following either oral or intravenous dosing of the prodrug to a human or animal subject, compared to the exposure obtained from dosing an equimolar amount of the drug of formula (IIIa) or (IIIb) itself. In an embodiment the exposure of the drug of formula (IIIa) or (IIIb) following dosing of the prodrugs is >20%, or >30%, or >40%, or >50%, or preferably >70% compared to the exposure obtained from dosing an equimolar amount of the drug of formula (IIIa) or (IIIb) itself. The pharmacokinetics of particular Examples prodrugs is shown in Table 4.

TABLE 4
Pharmacokinetics

% exposure of carabersat after dosing the
prodrugs of the invention via:

| Example | Oral dosing (po) | Intravenous dosing (iv) |
|---|---|---|
| 1 | 61% | 96% |
| 2 | 166% | 97% |
| 3 | 56% | 41% |
| 5 | 113% | 75% |
| 6 | 40% | 88% |
| 8 | 91% | 36% |

Solubility

In an embodiment prodrugs of the present invention are suitable for oral administration. The skilled person understands that the pH of the gastrointestinal tract changes along its length. For example, the stomach has a pH of around pH 1.5 and the GI tract after the stomach has a pH of around 5 to 7.5. For more detail see, for example, Measurement of gastrointestinal pH profiles in normal ambulant human subjects, Gut. 1988 August; 29(8): 1035-1041. Improved solubility is expected to result in improved absorption, and therefore improved oral bioavailability. Thus improved solubility at any pH value between around pH 1.5 to 8 is expected to improve oral bioavailability. Compounds of the invention were assessed for solubility in aqueous solutions having a pH of from 2 to 10. In an embodiment prodrugs of the invention have a solubility of >0.5 mg/mL in an aqueous solution having a pH of from 2 to 8. In an embodiment prodrugs have a solubility of >5.0 mg/mL, or >10.0 mg/mL, >100.0 mg/mL, or >200.0 mg/mL. In an embodiment the prodrugs have the aforementioned aqueous solubility at a pH within the range of from 4 to 8, or from 6 to 8.

In an embodiment prodrugs of the invention are administered intravenously. High prodrug solubility is advantageous in order to reduce the volume of solution administered to the patient, and to reduce the risk of damage to the circulatory system. Solubility of >10 mg/mL is preferred. Yet more preferred is solubility of >30 mg/mL or >100.0 mg/mL. Yet more preferred is solubility of >200.0 mg/mL. The solubility is measured in an aqueous solution having a pH of from 2 to 10, which pH range is advantageous for intravenous prodrug delivery. See, for example, A guide on intravenous drug compatibilities based on their pH, Nasser S C et al./Pharmacie Globale (IJCP) 2010, 5 (01)). In an embodiment the prodrugs of the claimed invention have solubility of >10 mg/mL in an aqueous solution having a pH of from 2 to 10. The solubility of certain Examples is shown in Table 5.

TABLE 5
Solubility

| Example | Solubility | |
|---|---|---|
| 1 | ~10 | mg/mL (pH 7.0) |
| 2 | >10 | mg/mL(pH 4.0) |
| 3 | <1 | mg/mL(pH 3.6) |
| 4 | >10 | mg/mL (pH 4.2) |
| 5 | >5 | mg/mL (pH 5.1) |
| 6 | >10 | mg/mL (pH 6.1) |
| 7 | <1 | mg/mL (pH 3.5) |
| 8 | ~10 | mg/mL (pH 4.0) |

1. A compound of formulae (Ia), (Ib), or a pharmaceutically acceptable salt thereof: 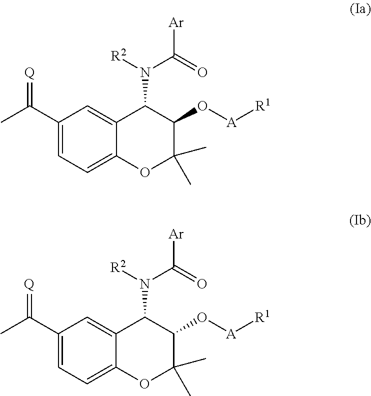
wherein in formula (Ia):
Ar is a monocyclic 5 or 6-membered heteroaryl ring or a phenyl ring, either of which is optionally substituted with one or more substituents selected from hydrogen, fluoro, chloro and iodo; and
wherein in the compounds of formula (Ib):
Ar is a monocyclic 5 or 6-membered heteroaryl ring optionally substituted with one or more substituents selected from hydrogen, fluoro, chloro, and iodo, or a phenyl group of formula (IIa): 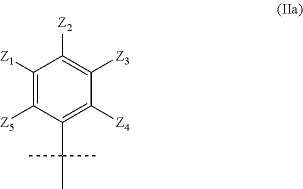
wherein Z₁, Z₂, Z₃, Z₄, and Z₅, are each independently selected from hydrogen fluoro, chloro and iodo, provided that at least one of Z₁ to Z₅ is iodo, and/or at least one of Z₄ and Z₅ is other than hydrogen; and
wherein in the compounds of formula (Ia) and (Ib):
Q is oxygen;
R² is hydrogen;
A is a direct bond, —C(O)O*—, —C(O)NH*, —C(R³)(R⁴)O*—, —C(O)O—C(R³)(R⁴)O*—, or —C(R³)(R⁴)O—C(O)O*— wherein the atom marked * is directly connected to R¹,
R³ and R⁴ are selected independently from H, fluoro, C₁₋₄ alkyl, and C₁₋₄ fluoroalkyl, or R³ and R⁴ together with the atom to which they are attached form a cyclopropyl group;
R¹ is selected from the group [1] through [18] wherein the atom marked ** is directly connected to A: 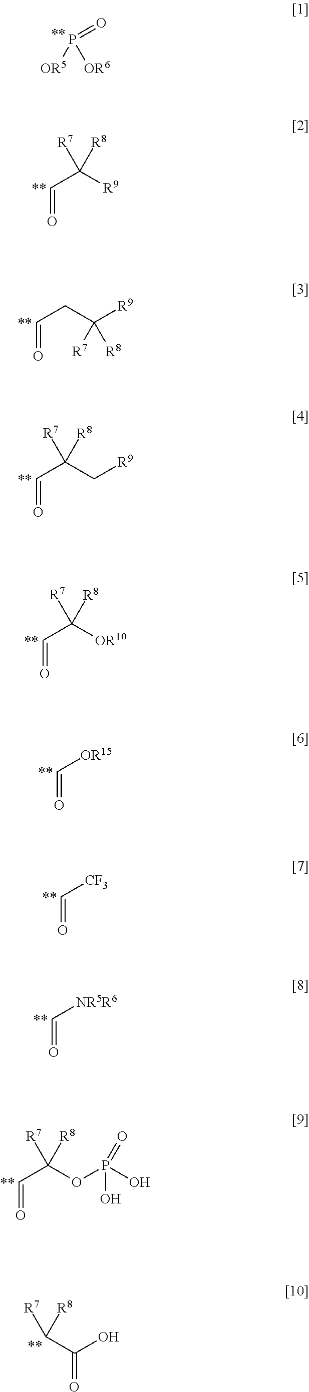 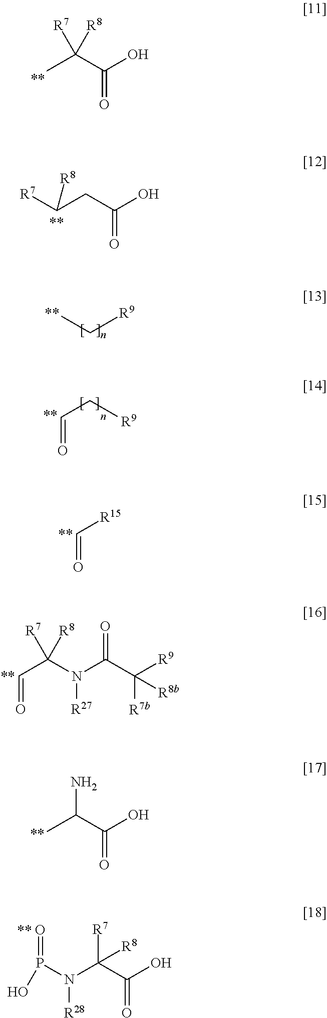
n is 0, 1, 2, or 3,
R⁵ and R⁶ are independently selected from H, C₁₋₄ alkyl, C₁₋₄ fluoroalkyl, —CH₂CH(OH)CH₂OH, —CH₂CH₂R⁹, and benzyl;
R⁷ and R^{7b} are independently selected from H, C₁₋₄ alkyl, and C₁₋₄ fluoroalkyl;
R⁸ and R^{8b} are independently selected from:
(i) H, C₁₋₄ alkyl, or C₁₋₄ fluoroalkyl, and
(ii) the side chain of a natural or unnatural alpha-amino acid
or R⁷ and R⁸ together with the atom to which they are attached form a C₃₋₇ carbocyclic ring;
R⁹ is selected from hydrogen, —N(R¹¹)(R¹²), or —N⁺(R¹¹)(R¹²)(R¹³)X⁻, —N(R¹¹)C(O)R¹⁴, —SO₃H, and —OP(O)(OH)₂,
wherein R¹¹, R¹², and R¹³ are independently selected from hydrogen, C₁₋₄ alkyl, and C₁₋₄ fluoroalkyl, or
R¹¹ and R¹² together with the nitrogen atom to which they are attached form a 3-8 membered heterocyclic ring optionally substituted with one or more substituents selected from hydrogen, fluoro, C₁₋₄ alkyl, C₁₋₄ fluoroalkyl, C₁₋₄ alkoxy, and —C(O)R₃;
or in the case where R¹ is group [16], and R⁹ is —NR¹¹R¹², and R¹¹ is hydrogen, C₁₋₄ alkyl, or C₁₋₄ fluoroalkyl, and R¹² is C₁₋₄ alkyl, or C₁₋₄ fluoroalkyl, then R¹² may join together with R^{8b} such that R¹² and R^{8b} together with the nitrogen to which R¹² is attached form a 5 or 6 membered cyclic amine group,
R¹⁴ is hydrogen, C₁₋₄ alkyl, or C₁₋₄ fluoroalkyl;
R¹⁰ is C₁₋₄ alkyl or C₁₋₄ fluoroalkyl, and R¹⁵ is independently selected from C₁₋₄ alkyl, C₁₋₄ fluoroalkyl, 3-pyridyl and 1,4-dihydro-1-methyl-pyridin-3-yl;
R²⁷ is selected from hydrogen, C₁₋₄ alkyl, and C₁₋₄ fluoroalkyl;
R²⁸ is selected from hydrogen, C₁₋₄ alkyl, and C₁₋₄ fluoroalkyl;
X⁻ is a pharmaceutically acceptable anion.

2. A compound as claimed in claim 1 wherein Ar is optionally substituted thiophenyl.

3. A compound as claimed in claim 1 of formula (Ib) wherein Ar is selected from the group consisting of:

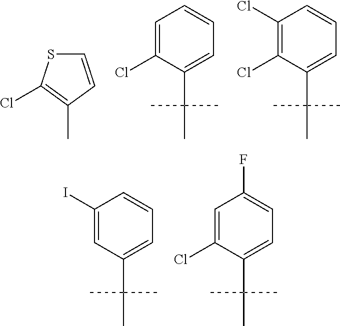

4. A compound as claimed in claim 1 of formula (Ia) wherein Ar is selected from the group consisting of:

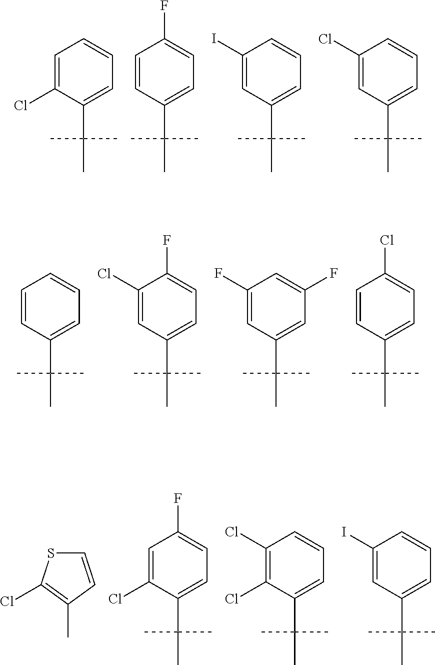

5. A compound as claimed in claim 1 wherein A is a direct bond or C(R³)(R⁴)O*—.

6. A compound as claimed in claim 1, wherein R³ and R⁴ are both H, or R³ and R⁴ are both C₁₋₄ alkyl, or R³ is H and R⁴ is C₁₋₄ alkyl.

7. A compound as claimed in any claim 1, wherein R¹¹, R¹², and R¹³ are independently methyl or ethyl.

8. A compound as claimed in any claim 1, wherein R¹¹ and R¹² together with the nitrogen atom to which they are attached form a 5 or 6 membered cyclic amino group.

9. A compound as claimed in claim 1 wherein R⁷ is hydrogen and R⁸ is the side chain of a natural or unnatural amino acid.

10. A compound as claimed in claim 1 wherein R^{7b} is hydrogen and R^{8b} is the side chain of a natural or unnatural amino acid.

11. A compound as claimed in claim 1 wherein the side chain of the natural or unnatural amino acid is selected from —CH(CH₃)₂, —(CH₂)₃CH₂NH₂, —CH(CH₃)(CH₂CH₂CH₃), —CH₂CH(CH₃)₂, —CH₂OH, and the histidine side chain:

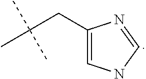

12. A compound as claimed in claim 1, wherein R⁷ and R⁸ are both hydrogen.

13. A compound as claimed in claim 1, wherein R^{7b} and R^{8b} are both hydrogen.

14. A compound as claimed in claim 1 wherein R⁶ is selected from —CH₂CH(OH)CH₂OH, —CH₂CH₂NR¹¹R¹², and —CH₂CH₂NR¹¹R¹²R¹³X⁻.

15. A compound as claimed in claim 1 wherein R⁵ and R⁶ are hydrogen.

16. A compound as claimed in claim 1 wherein R¹ is selected from any one of the following groups:

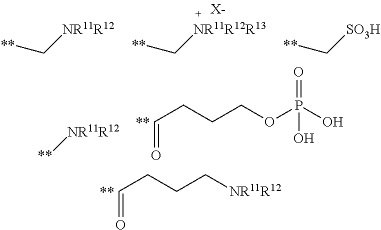

17. A compound as claimed in claim 1, wherein -A-R¹ is selected from the following, wherein the atom marked ** is directly connected to the oxygen atom:

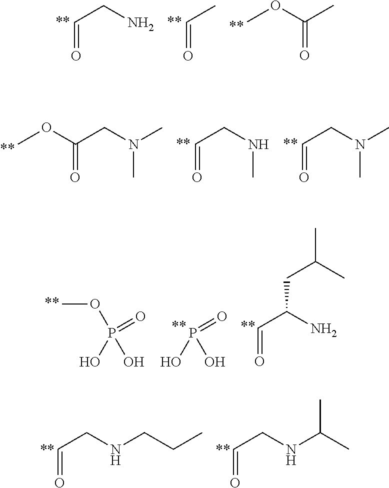

18. A compound as claimed in claim 1 wherein A is a direct bond and R¹ has the formula (7A): 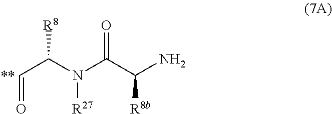
wherein R²⁷ is hydrogen or C₁₋₄ alkyl; and R⁸ and R^{8b} are each independently the side chain of a natural alpha-amino acid.

19. A compound as claimed in claim 18 wherein R⁸ and R^{8b} are each independently selected from methyl, isopropyl and —CH₂CH(CH₃)₂.

20. A compound as claimed in claim 18, wherein R²⁷ is hydrogen or methyl.

21-30. (canceled)
